(19) United States
(12) Patent Application Publication
(10) Pub. No.: US 2002/0143169 A1
(43) Pub. Date: Oct. 3, 2002

(54) Gene related to carcinogenesis and spermatogenesis and method for using same

(76) Inventors: Molly F. Kulesz-Martin, Portland, OR (US); Yuangang Liu, Portland, OR (US)

Correspondence Address: DUNN & ASSOCIATES, P.O. Box 10, NEWFANE, NY, 14108, US

(21) Appl. No.: 09/850,697

(22) Filed: May 8, 2001

Related U.S. Application Data
(63) Continuation-in-part of application No. 09/777,753, filed on Feb. 6, 2001.
Application No. 09/777,753 is a continuation-in-part of application No. 08/938,308, filed on Sep. 27, 1997, now abandoned.

(51) Int. Cl. C12Q 1/68; G01N 33/574; C07H 21/04; C07K 16/30

(52) U.S. Cl. 536/23.2; 435/6; 530/388.8; 435/7.23

(57) ABSTRACT

Using RNA differential display in search of altered gene expression in a cloned cell model of chemical carcinogenesis, a novel spermatogenesis associated factor (SPAF) was isolated that is altered and overexpressed in a poorly differentiated squamous cell carcinoma. Both Northern analysis of the normal tissue distribution of SPAF revealed that SPAF is predominantly expressed in testis. Further analysis by immunohistochemical staining with anti-SPAF antibody localized the SPAF protein in the spermatogonia and primary spermatocytes up to early pachytene stage. This highly selective expression makes it ideal as a putative molecular marker for diagnosis and treatment efficacy of human conditions such as infertility and cancer, as well as a potential target for therapy. This application claims the use of SPAF protein, peptide, mRNA and cDNA for the design of drugs for prevention and treatment of abnormal development and tumors, for fertility/infertility/contraception and for the use of SPAF cDNA sequence or anti-SPAF antibodies in the diagnosis of human cancer and human infertility.

This is a Continuation-in-Part of U.S. patent application Ser. No. 09/777,753 filed Feb. 6, 2001, which is a Continuation-in-Part of U.S. Ser. No. 08/938,308 filed Sep. 27, 1997.

This invention was made under a grant from the U.S. National Institute of Health (NIH), Grant NIH R01-CA31101. The United States Government may have certain rights in the invention.

### BACKGROUND OF THE INVENTION

This invention relates to genes involved in cell growth reproduction. The invention is especially concerned with genes involved in malignant growth and other cellular reproduction.

Until the present time, very few, if any, genes involved in cellular reproduction, especially in higher animals, have been identified and isolated. Further cDNA complementary forms of such genes, and in particular those associated with both malignant and normal cellular growth, have been relatively unknown. Translated proteins from such genes or antibodies thereto are similarly unknown and uncharacterized.

### BRIEF DESCRIPTION OF THE INVENTION

Using RNA differential display in search of altered gene expression in a cloned cell model of chemical carcinogenesis, a novel spermatogenesis associated factor (SPAF) was isolated that is altered and overexpressed in a poorly differentiated squamous cell carcinoma. Both Northern analysis of the normal tissue distribution of SPAF revealed that SPAF is predominantly expressed in testis. Further analysis by immunohistochemical staining with anti-SPAF antibody localized the SPAF protein in the spermatogonia and primary spermatocytes up to early pachytene stage. This highly selective expression makes it ideal as a putative molecular marker for diagnosis and treatment efficacy of human conditions such as infertility and cancer, as well as a potential target for therapy. This application claims the use of SPAF protein, peptide, mRNA and cDNA for the design of drugs for prevention and treatment of abnormal development and tumors, for fertility/infertility/contraception and for the use of SPAF cDNA sequence or anti-SPAF antibodies in the diagnosis of human cancer and human infertility.

This invention is directed to purified SPAF DNA sequences, SPAF cDNA sequences, SPAF proteins, anti-SPAF antibodies, a method for diagnosis of cancer by contacting a cell with a probe comprising SPAF cDNA and detecting a reaction, a method for isolating SPAF gene comprising contacting the gene with SPAF cDNA and isolating a resulting conjugate, a method for diagnosis of cancer comprising contacting a cell with anti-SPAF antibody and detecting a reaction to the antibody, a method for diagnosing infertility by contacting testes generated cellular material with anti-SPAF antibody to determine the concentration of SPAF, a method for isolation of SPAF gene by contacting the gene with anti-SPAF antibody and isolating the resulting conjugate, a chemotherapeutic reagent comprising an antibody to SPAF protein, a chemotherapeutic reagent comprising an antibody to SPAF protein conjugated with a tumor inhibiting material, and a viral or plasmid vector containing a testes specific nucleic acid comprising at least a portion of SPAF.

### BRIEF DESCRIPTION OF THE DRAWINGS

FIG. 1 is a gel RNA differential display comparing 291, O3C, O3RAT and O9RAT. Altered gene expression is detected by the RNA differential display. The murine epidermal cell lines indicated were cultured until 70% confluent. Cells were harvested and purified, and total RNA's were subjected to reverse transcription followed by PCR amplification using primer set AP7/T₁₁G in the presence of ³⁵S-dATP. The PCR products were fractionated and displayed on a 5% polyacrylamide sequencing gel. Positions of differentially displayed products designated 7G1 and 7G2 are shown.

FIG. 2 is a Northern analysis gel of differential display products in the cloned cell model of carcinogenesis comparing 291, O3C, O3R, O5R and O9R. Ten μg of total RNA from each cell type shown was separated by 1.2% agarose formaldehyde gel electrophoresis. The blots were hybridized with ³²P-labeled cloned differential display products a) 7G1 and b) 7G2. Fold increases in signal intensity relative to 291 are shown, adjusted to 7S loading control.

FIG. 3 is a gel showing expression of 7G2 in normal mouse tissues. Ten μg of total RNA from the adult mouse tissues indicated were separated in a 1.2% agarose formaldehyde gel. The RNA's from 291 cells and O3RAT cells were included as negative and positive control, respectively. The blot was hybridized with ³²P-labeled cDNA 7G2 fragments isolated from RNA differential display. 7S RNA hybridization is shown as RNA loading control.

FIG. 4 shows a nucleotide sequence of partial SPAF cDNA identified by RNA differential display. Flanking sequences representing primer sets used for PCR and a putative polyadenylation signal are underlined.

FIG. 5 shows the nucleotide and predicted amino acid sequence of SPAF. A putative polyadenylation signal is underlined. The translated sequence is indicated by single letter amino acid designations where A=Ala, C=Cys, D=Asp, E=Glu, F=Phe, G=Gly, H=His, I=Ile, K=Lys, L=Leu, M=Met, N=Asn, P=Pro, Q=Gln, R=Arg, S=Ser, T=Thr, V=Val, W=Trp, and Y=Tyr. Bold letters indicate the AAA-protein family signatures.

FIG. 6 is an electrophoresis gel showing production of his-tagged SPAF fusion protein. The N-terminal portion of SPAF (60-397 a.a) was fused in frame to a poly-histidine tag in plasmid pProEX™HTb (BRL/GIBCO). The fusion protein produced in E.coli was induced by adding 0.6 mM IPTG (isopropyl-β-D-thiogalactopyranoside) to exponentially growing cultures. whole-cell extracts of E.coli were applied to a nickel affinity column and the bound fusion protein was eluted with eluent solution containing 100 mM imidizol. Lysates from cultures uninduced and IPTG induced at 1, and 3 hours, alone with 10 μl of fusion protein eluted from the nickel column (E1, E2) were subjected to electrophoresis through a 10% SDS-polyacrylamide gel, and stained with Coomassie blue. Bovine serum albumin (BSA) was included as quantitation control.

FIG. 7 is an SDS polyacrylamide gel showing immunoprecipitation of in vivo translated SPAF protein by anti-SPAF anti-serum. SPAF1 protein and luciferase reference protein were in vitro translated in the presence of [³⁵S]methionine and subjected to immunoprecipitation with SPAF anti-serum (SpAb) and corresponding pre-immunoserum (Pre). The immunocomplexes were analyzed by SDS-polyacrylamide gel electrophoresis and autoradiography.

FIG. 8A shows constructs of plasmids containing SPAF cDNA's, 1 to 2094 (SPAF); 106 to 2094 (SPAF1); 234 to 2094 (SPAF2). The molecular weight of SPAF's by calculation from deduced amino acid sequences are parenthesized.

FIG. 8B shows a 7.5% SDS-polyacrylamide gel through which proteins from the plasmids of FIG. 8A transcribed/translated in vitro in the presence of [³⁵S] methionine, were electrophoresed and visualized by autoradiography.

FIG. 8C shows a size comparison of in vitro translated SPAF proteins with endogenous SPAF protein by immunoblotting with anti-SPAF polyclonal antibodies. In vitro translated SPAF proteins were enriched by immunoprecipitation and subjected to immunoblotting analysis along with testis lysate from adult mice.

FIG. 9 shows an amino acid sequence comparison of SPAF, S. acidocaldarius SAV (L17042), rat p97ATPase (U11760), and S. cerevisiae cdc48 (X56956). Sequences were aligned using the GCG Pileup program. The ATPase modules are delineated in brackets. Motifs A and B of the consensus sequence for ATP binding are overlined. The putative cdc2 phosphorylation sites are marked by asterisks.

FIG. 10 shows a Northern blot analysis of the expression of SPAF during spermatogenesis. Ten μg of each total RNA from 1-, 2-week old and adult mouse testes. and adult epididymis was loaded on 1.2% agarose formaldehyde gel. The total RNA's from 291 and O3RAT were included as positive and negative controls respectively. The blots were hybridized with ³²P-labeled full length cDNA fragment of SPAF. A 7S RNA hybridization is shown as control for RNA loading.

FIG. 11 shows a Western analysis of the expression of SPAF protein during spermatogenesis. Twenty micrograms of each lysate from adult epididymis and testes dissected from 1-, 2-week old and adult mice was fractionated in a 7.5% SDS-polyacrylamide minigel and transferred to a nitrocellulose membrane. The blot was incubated with the SPAF antibody followed with protein-A conjugated horseradish peroxidase. The immuno-complexes were visualized by chemiluminescence.

FIG. 12A shows a paraffin section from adult mouse testis at 100 magnification, immunostained with affinity purified anti-SPAF antibody (SpAb). Paraffin sections from adult mouse testis (a, c), and 2 week old testis (d), were immunostained with affinity purified anti-SPAF antibody (SpAb). The adult testis section was immunostained with preimmune (PreI) as negative control (b). The section was deparaffined, permeabilized in 0.3% Triton-X-100/PBS for 10 min, and incubated with 0.1 μg/ml SpAb or 1 μg/ml preimmune antibody overnight at 4° C.

FIG. 12B shows a paraffin section from adult mouse testis at 100 magnification immunostained with preimmune (PreI) as a negative control. Paraffin sections from adult mouse testis (a, c), and 2 week old testis (d), were immunostained with affinity purified anti-SPAF antibody (SpAb). The adult testis section was immunostained with preimmune (PreI) as negative control (b). The section was deparaffined, permeabilized in 0.3% Triton-X-100/PBS for 10 min, and incubated with 0.1 μg/ml SpAb or 1 μg/ml preimmune antibody overnight at 4° C.

FIG. 12C is similar to FIG. 12A, except at 400 magnification, letters: A, type A spermatogonia; L, leptotene spermatocyte; Z, zygotene spermatocyte; D, spermatid; S, Sertoli cell. Magnification: a, b 100-fold; c, d, 400-fold.

FIG. 12D shows a paraffin section from a 2 week old testis. Paraffin sections from adult mouse testis (a, c), and 2 week old testis (d), were immunostained with affinity purified anti-SPAF antibody (SpAb). The adult testis section was immunostained with preimmune (PreI) as negative control (b). The section was deparaffined, permeabilized in 0.3% Triton-X-100/PBS for 10 min, and incubated with 0.1 μg/ml SpAb or 1 μg/ml preimmune antibody overnight at 4° C.

FIG. 13 shows Western analysis of the expression of SPAF protein in the cloned epidermal cell model of carcinogenesis. Whole cell lysates were isolated from 70% confluent cultures of 291, O3C, O3RAT, O5RAT, and O9RAT. Twenty micrograms of lysates quantitated by Protein-Assay (Bio-Rad) was fractionated in a 7.5% SDS-polyacrylamide minigel, and transferred to a nitrocellulose membrane. The blot was incubated with the SPAF antibody followed with protein-A conjugated horseradish peroxidase (Amersham). The immuno-complexes were visualized by chemiluminescence (Pierce).

FIG. 14 shows a 1% agarose electrophoresis gel showing PCR amplified human SPAF homologue visualized by ethidium bromide staining.

FIG. 15 shows a preliminary amino acid sequence of human SPAF protein.

FIG. 16 shows a Northern blot of human SPAF gene expression in various normal human tissues.

FIG. 17 shows an identifying nucleotide sequence of human SPAF (hSPAF) as requested in Genbank Accession number AF361489.

FIG. 18 shows a more detailed and reliable deduction of an identifying amino acid sequence of human SPAF protein in comparison with a major portion of the preliminary amino acid sequence of FIG. 15 where A=Ala, C=Cys, D=Asp, E=Glu, F=Phe, G=Gly, H=His, I=Ile, K=Lys, L=Leu, M=Met, N=Asn, P=Pro, Q=Gln, R=Arg, S=Ser, T=Thr, V=Val, W=Trp, and Y=Tyr. Due to large blocks of identity, it is apparent that the preliminary suggested amino acid sequence and the actual amino acid sequence both represent the human SPAF protein due to about 93% correspondence from amino acid 1 through amino acid 800.

FIG. 19 shows the more detailed and reliable amino acid sequence of human SPAF protein using three letter amino acid designations.

FIG. 20 is a schematic illustration of genomic structure in SPAF/FGF-2 locus.

FIG. 21 shows analysis of FGF-2 expression in an epidermal model of carcinogenesis by semi-quantitative RT-PCR using primer sets for FGF-2. Amplification of hprt (left panel) was included as loading control.

### DETAILED DESCRIPTION OF THE INVENTION

Normal mouse skin keratinocytes were initiated with dimethylbenz[α]anthracene and variant clones with benign or malignant phenotypes were derived using procedures described by Kulesz-Martin, et al., 1988, Carcinogenesis, 9, 171-174. To identify the specific genes which were altered during the multistage process of mouse skin carcinogenesis, the gene expression patterns of the normal parental epidermal cell, an initiated epidermal cell, a benign papilloma, and a poorly differentiated squamous carcinoma were compared by using RNA differential display as described by Liang, et al., 1992, Science 257, 967-97. A gene that is altered and overexpressed in a poorly differentiated squamous carcinoma was identified. A Northern analysis of the tissue expression pattern of the gene revealed testis-specific expression. By screening a mouse testis cDNA library combined with ligation-anchored polymerase chain reaction, a 2894 bp cDNA was cloned which contains an open reading frame encoding a 892-amino acid protein. A search of nucleotide and protein databases indicates that this is a novel gene. Sequence motifs suggest that it is a new member of the AAA-protein family (ATPase Associated with diverse cellular Activities) as described by Confalonieri, et al., 1995, Bioessays, 17, 639-650. The ATP-binding domain region of the new gene shares about 70% similarities with corresponding regions of other members in this family such as cdc48, a yeast cell cycle protein involved in spindle pole body formation and endoplasmic reticulum fusion as described by Latterich, et al., 1995, Cell, 82, 885-893 and Frohlich, et al., 1991, Journal of Cell Biology, 114, 443-453, and p97, an ATPase from rat that functions in rebuilding Golgi cisternae after mitosis [Acharya, et al., 1995, Cell, 82, 895-904 and Rabouille, et al., 1995, Cell, 82, 905-914]. But both N-terminal and C-terminal regions of this novel gene show no significant similarities with those of other proteins in this family, suggesting that it has unique physiological functions. Immunohistochemical staining of mouse testis tissue sections using a polyclonal antibody raised against the N-terminal portion of this protein revealed a unique expression pattern in seminiferous tubules. High level expression of this protein was restricted to the spermatogenic cells in the early stage of spermatogenesis including spermatogonia and primary spermatocytes up to the early pachytene stage. This expression pattern was confirmed by Northern analysis (a convention technique to analyze mRNA level using a sequence specific DNA fragment which is labeled with radioisotope) and Western analysis (a conventional technique to analyze protein level using specific antibody as probe) of SPAF expression in testes samples taken at different development stages. Therefore, this novel gene is designated as SPAF for spermatogenesis associated factor. This is the first gene of the AAA-protein family found to be associated with germ cell development and the first to be associated with malignant transformation. The SPAF protein is one of very few markers (a well characterized one is c-kit [Sorrentino, et al., 1991, Oncogene, 6, 149-151], a receptor of steel factor) of early germ cell development and provides a potentially very useful marker of this process and these cells. The presence of SPAF in malignant cells has potential for applications, not only in basic research on carcinogenesis and differentiation, but also in cancer diagnosis and the design of selective cancer therapy by targeting this gene product.

### EXAMPLES

### Cell Culture and Reagents

Normal 291 epidermal cells were grown in Eagle's minimal essential medium (a commonly used medium for the growth of mammalian cells provided by GIBCO/BRL, Grand Island, N.Y., composition shown in Table 2) with Earle's salts, composition shown in Table 3, without CaCl₂ (a balanced salt solution for the optimization of the growth condition provided by GIBCO/BRL), supplemented with 5% (v/v) fetal calf serum treated with chelex-100 resin (a weak cation chelating resin for depleting calcium ion in the serum provided by Bio-Rad, Hercules, Calif.) to reduce calcium concentration, and 10% (v/v) mouse dermal fibroblast conditioned medium (culture medium incubated 2 to 3 days with mouse derm primary fibroblast cultures), 10 ng/ml epidermal growth factor (UBI), 1% (v/v) antibiotic-antimycotic (10,000 units of penicillin, 10,000 μg of streptomycin, and 25 μg of amphotericin B) (GIBCO/BRL) and 0.02 mM CaCl₂ (low calcium) [Kulesz-Martin, et al., 1988, supra]. The initiated cell lines (O3C, O5C, and O9C) and the tumorigenic cell lines (O3RAT, O5RAT, and O9RAT) were maintained in Eagle's minimal essential medium supplemented with 5% (v/v) fetal calf serum, 10 ng/ml epidermal growth factor (UBI), 1% (v/v) antibiotic-antimycotic (10,000 unites of penicillin, 10,000 μg of streptomycin, and 25 μg of amphotericin B) and 1.4 mM Ca²⁺ (high calcium). For differential display, all cell lines were compared under the low calcium conditions required for 291 cell growth.

### RNA Differential Display

Differential display was performed as described [Liang, et al., 1992, supra] with minor modifications. Briefly, reverse transcription-polymerase chain reactions (RT-PCR) were performed in duplicate using 0.2 μg RNA from two independent cell preparations, 2 μM dT,₁₁ 3′ anchor primer (5′-AAGCTTTTTTTTTTTG-3′) (SEQ. I.D. #1) (Genhunter, Brookline, Mass.), 250 μM deoxynucleotide triphosphate, 10 units of Rnasin, a common Rnase inhibitor (available from Promega, Madison, Wis.; stratagene and GIBCO/BRL), and 100 units of Moloney murine leukemia virus reverse transcriptase (GIBCO/BRL), followed by PCR using the same anchor primer and a randomly designed twelve arbitrary 5′primer, 5′-AAGCTTAAGCAGG-3′ (SEQ. I.D. #2) (Genhunter) in the presence of 10 μCi α-[³⁵S]dATP (Amersham, Arlington Heights, Ill.) with 40 cycles, each consisting of 94° C. for 30 sec, 40° C. for 2 min, and 72° C. for 30 sec. The amplified cDNA's were fractionated through 5% polyacrylamide sequencing gels and exposed to X-ray film for autoradiography.

### Recovery and Selection of Positive Clones

The differentially displayed RT-PCR products were excised from the dried gels. Their cDNA's were recovered and reamplified by PCR under the conditions described above except that no radioisotope was used. The cDNA's confirmed by Northern analysis were cloned into pBluescript (KS), a commonly used plasmid for general cloning (Stratagene, La Jolla, Calif.) treated with EcoRV and Taq polymerase. The ligation mixture was used to transform E.coli DH5α (GIBCO/BRL). Equivalent to E. coli JM 101 (ATCC Deposit No. 47015). Plasmids were extracted using a Qiagen plasmid miniprep kit (Qiagen, Santa Clarita, Calif.), in brief, the bacteria were lyzed in lysis buffer (200 mM NaOH, 1% sodium dodecyl sulfate) and neutralized by 3 M potassium acetate [pH5.5]. After centrifugation, the supernatant was loaded to a column filled with glass beads. After wash with wash buffer (1 M NaCl, 50 mM [N-morpholino]propane-sulfonic acid [pH7.0], 15% ethanol), the plasmid was eluted from the column by TE buffer (10 mM Tris-HCL, [pH8.0], 1 mM ethylenediaminetetraacetic acid). The purified plasmids were then subjected to dot-blot selection [Callard, et al., 1994, Biotechniques, 16, 1096-1102]. HindIII restriction fragments of the representative clones were radiolabelled and used as probes in Northern analysis to select true positives, i.e. those which matched the patters of expression observed by RT-PCR.

### RNA Preparation and Northern Blot Analysis

Total RNA was extracted from cells at approximately 70% confluence with Trizol Reagent (GIBCO/BRL). The cells in a 150 mm plate were homogenized with 1 ml of Trizol Reagent and 0.2 ml of 24:1 (v/v) chloroform/isoamyl alcohol. After 10 min centrifugation at 10,000×g, the supernatant was precipitated with equal volume isopropanol. The precipitant was collected by centrifugation at 10,000×g for 10 min. The RNA pellet was dissolved in H₂O. Tissue samples were harvested from adult male mice and homogenized in Trizol Reagent using a Polytron (a homogenizer manufactured by Kinematica GmbH, Germany). Ten μg of total RNA per cell type was separated in a 1.2% agarose, 1 M formaldehyde gel and transferred to nylon membrane (Schleicher and Schuell, Keene, N.H.) according to standard protocols [Sambrook, et al., 1989, Molecular Cloning, Cold Spring Harbor Laboratory]. DNA fragments from RNA differential display and 7S RNA (a ribosomal RNA used for loading control in Northern analysis) [Balmain, et al., 1982, Nucleic Acids Research, 10, 4259-4277] were labeled with [³²P] deoxycytosine triphosphate by random-priming. The membrane was hybridized with [³²P]deoxycytosine triphosphate-labeled probe in hybridization buffer (50% formamide, 5×SSPE (0.75 M NaCL, 50 mM NaH₂PO₄, 5 mM EDTA), 10×Denhart's (0.2% Ficoll 400 (a nonionic synthetic polymer of sucrose), 0.2% polyvinylpyrrolidone, 0.2% bovine serum albumin), 0.2% sodium dodecyl sulfate, and 100 μg/ml salmon sperm DNA) at 42° C. overnight. The membranes were washed twice in 2×SSC (0.3 M NaCL, 30 mM sodium citrate [pH7.0]) and 0.1% sodium dodecyl sulfate for 30 min at 60° C. and exposed to X-ray films for 1 hour at room temperature for 7S RNA and 2 days at −80° C. for all others. For rehybridization, membranes were subjected to 0.5×SSC (75 mM NaCL, 7.5 mM sodium citrate [pH7.0]) containing 0.1% sodium dodecyl sulfate and 20 mM ethylenediaminetetraacetic acid for 10 min at 100° C. Quantification was performed by scanning the autoradiograms by means of a Computing Densitometer (Molecular Dynamics, Sunnyvale, Calif.) and analyzed using ImageQuant software (Molecular Dynamics).

### cDNA Cloning

A mouse testis cDNA library (Stratagene) constructed using the uni-Zap vector (a bacterial phage vector used in construction of mouse testis cDNA library constructed by Stratagene) with poly-deoxythymidine-primed cDNA inserts was screened using a [α-³²P] deoxycytosine triphosphate-labeled partial cDNA fragment (7G2) obtained from RNA differential display. DNA probe was prepared by random of the partial cDNA fragment using [α-³²P] deoxycytosine triphosphate (Amersham, Arlington Heights, Ill.) following manufacturer's protocols. SPAF cDNA fragment in pBluescript SK-vector (pBSPAF), GenBank Accession No. 52324, was excised from positive known uni-Zap phage isolated from a mouse testis cDNA library by co-infection with the known filamentous helper phage (a bacterial phage used for excision of plasmid from uni-Zap vector) (Stratagene). For a complete description of uni-Zap and filamentous helper phage see Nucleic Acids Research, 16: 7583-7597, 1988. The 5′-end of SPAF was amplified by ligation-anchored polymerase chain reaction [Troutt, et al., 1992, Proceedings of Nat. Acad. of Sciences U.S.A., 89, 9823-9825]. Two oligonucleotide primers complementary to SPAF mRNA were synthesized: 21-mer (I; 5′-TTCCTGAGCCTGAAGCACAGC-3′) (SEQ. I.D. #3) and 28-mer (II; 5′-CATAGTGTTAAGTCCAAGATGAACAAGG-3′) (SEQ. I.D. #4) representing antisense sequences corresponding to 471-451 bp and 267-240 bp, respectively, of SPAF cDNA. The ligation-anchored PCR were carried out using the Marathon cDNA Amplification Kit (Clontech, Palo Alto, Calif.) following the manufacturer's manual. In brief, 1 μg of mRNA purified from mouse testis was incubated with 1 μl of 21-mer primer, 2 μl of 5×reverse transcription buffer (125 mM Tris-Cl, pH 8.3, 188 mM KCl, 7.5 MM MgCl₂ and 25 mM dithiothreitol), 1 μl of 10 mM deoxynucleotide triphosphate, 100 U of Moloney murine leukemia virus reverse transcriptase in a total volume of 10 μl at 42° C. for 1 hour. The second cDNA strand was synthesized by addition of 4 μl of second-strand reaction mixture (4 U Rnase H, 100 U DNA polymerase I), 1.6 μl of 10 mM deoxynucleotide triphosphate, and 48.4 μl of H₂O and incubation at 16° C. for 1 hour. After precipitation with ethanol, the cDNA was ligated to a synthetic adaptor

| | |
|---|---|
| 5′CTAATACGACTCACTATAGGGCTCGAGCGGCCGCCCGGGCAGGT3′) | (SEQ. I.D. #5) |

(SEQ. I.D. #5) by incubation of 5 μl of cDNA, 2 μl of the adaptor (10 mM), 1 U ligase, in ligation buffer (50 mM Tris-HCl [pH7.5], 10 mM MgCl₂, 10 mM dithiothreitol, 1 mM ATP) at 16° C. overnight. The 5′-cDNA end of SPAF was amplified with AP2 primer (5′ ACTCACTATAGGGCTCGAGCGGC3′) (SEQ. I.D. #6) and 28-mer in a mixture of 5 μl of ligated cDNA, 1 μl of AP2 primer (10 μM), 1 μl of 28-mer primer (10 μM), 5 μl of 10×PCR reaction buffer (100 mM Tris-Cl, pH 8.4, 500 mM KCl, 15 mM MgCl₂ and 0.01% gelatin), and 1 U pfu DNA polymerase (provided by Stratagene) in a Perkin-Elmer DNA Thermal Cycler programmed for 30 cycles of 94° C. for 30 sec, 63° C. for 1 min, and 72° C. for 2 min with 5 min extension at 72° C. The amplified products were gel purified using the QIAEX II gel extraction kit (Qiagen) as follows. The amplified DNA fragment excised from the agarose gel was extracted in high salt buffer and loaded in a spin column filled with glass beads. After washing with wash buffer (1 M NaCl, 50 mM [N-morpholino]propane-sulfonic acid [pH7.0], 15% ethanol), the plasmid was eluted from the column by TE buffer (10 mM Tris-HCL, {pH8.0], 1 mM ethylenediaminetetraacetic acid). The purified DNA was digested with NotI and ligated into pBluescript(KS) plasmid (Stratagene) digested with NotI and SmaI. The ligation mixture was used to transform E.coli strain XL1-Blue (Stratagene, equivalent to E. coli DH5α and E. coli JM101), and clones containing inserts were selected by blue-white color selection, and confirmed by PCR using the 28-mer and a sense primer (5′-GCCTCGCCCTCGCTCTCTGC-3′) (SEQ. I.D. #7) corresponding to bp 113-133 of SPAF cDNA. The full length SPAF cDNA was constructed by subcloning NotI and BstEII digested fragment into pBSPAF digested with the same enzymes.

### DNA Sequencing and Analysis

SPAF cDNA was digested with SacII and XhoI to generate 3′ and 5′ protruding ends, and digested with exonucleaseIII to generate serial deletions. A set of overlapping deletion clones were generated and sequenced with M13 reverse primer (5′ AGCGGATAACAATTTCACACAGG3′ (SEQ. I.D. #8) (GIBCO/BRL)) using an automatic DNA sequencer. DNA sequence analysis, including searches for homology, motifs and structure predictions, was performed using Genetics Computer Group programs (Wisconsin Package V.8.0).

### In vitro Translation

In order to define the true translational start site, protein products of SPAF cDNA's, 1 to 2904 (SPAF, the full length SPAF cDNA), 106 to 2904 (SPAF1, a truncated SPAF cDNA which is 106 base pair shorter than the full length SPAF, which encodes a putative polypeptide of 857 amino acids), 234 to 2904 (SPAF2, a truncated SPAF cDNA which is 228 base pair shorter than the full length SPAF, which encodes a putative polypeptide of 820 amino acids) were cloned into the NotI/XhoI, SacII/XhoI, and BamHI/XhoI sites respectively, of pBluescript KS under the T₇ phage promoter (a bacterial phage DNA sequence (5′TAATACGACTCACTATAGGG3′) (SEQ. I.D. #9) which initiates the transcription of its downstream DNA in the presence of T₇ RNA polymerase) respectively. The constructs were linearized with XhoI and transcribed with T₇ RNA polymerase using the mCAP™ RNA Capping Kit (Stratagene), a in vitro mRNA transcription system. The mRNA of SPAF's was synthesized by incubation of 1 μg of linearized SPAF plasmid as template with 10 U of T₇ RNA polymerase, 1 μl of 10 mM ribonucleotide triphosphate, 2.5 μl of 3 mM CAP analog, and 1 μl of 0.75 M dithiothreitol at 37° C. for 30 min. The transcripts were translated in the presence of ³⁵S-methionine (Amersham) according to standard protocols (Promega). 5 μl of each in vitro translated protein was fractionated on a 7.5% sodium dodecyl sulfate-polyacrylamide gel, and visualized by autoradiography.

### Production of SPAF-specific Antibodies

Polyclonal antibodies were raised against the unique N-terminal region of the novel SPAF sequence, upstream of the first ATP binding module as follows: a fragment of SPAF corresponding to amino acids 60-397 was cloned in-frame to the poly-histidine tag in plasmid pProEX™HTb a plasmid provided by GIBCO/BRL for the expression of engineered protein. It consists of a Trc promoter induced by isopropylthio-β-galactoside for high level expression, and an affinity tag of six histidines for protein purification through a nickel affinity column. The resultant plasmid was used to transform E.coli strain HB101 (GIBCO/BRL, ATCC Deposit No. 31343). Expression of the fusion protein was induced by adding 3 mM isopropyl-β-D-thiogalactopyranoside to exponentially growing cultures. The bacteria were homogenized in lysis buffer (10 mM Tris-HCl, pH8.3, 100 mM NaCl, 5 mM ethylenediaminetetraacetic acid, 1 mM phenymethylsulfonylfluoride) containing 0.1 mg/ml lysozyme, and by passing through a 22G syringe needle. Inclusion bodies were solubilized in 1% sarcosol (a detergent used for lysis of bacteria) and neutralized with 5% Triton-100 (a non-ionic surfactant). The solubilized protein was dialyzed in the lysis buffer and subjected to nickel affinity column purification. The bound protein was eluted with lysis buffer in the presence of 100 mM imidozol. The eluted fusion protein was dialyzed against phosphate-buffered saline and used for the generation of polyclonal antibodies by standard protocol. Three New Zealand white female rabbits were each injected subcutaneously with 200 μg of purified protein emulsified with equal volume of Freund's complete adjuvant (1 mg of heat-killed and dried Mycobacterium tuberculosis, 0.85 ml paraffin oil, and 0.15 ml mannide monooleate). One month after the primary injection, boost injections were repeated every 2 weeks with 150 μg of purified protein emulsified in incomplete Freund's adjuvant. Antisera were tested for their ability to precipitate in vitro translated SPAF protein. The polyclonal anti-SPAF antibodies (called SpAb) were affinity purified against 6 mg purified histidine tagged SPAF protein coupled to an Aminolink column, a column filled with 1,1′-carbonyldiimidazole activated agarose beads provided by Pierce. Six milligrams of SPAF fusion protein were incubated with 1 ml of activated agarose beads and 50 μl of reducing solution (10 mM NaOH, 6% w/v sodium cyanoborohydride) for 3 hours in a fume hood. After wash with 5 ml of phosphate buffered saline, the column was incubated with 2 ml of quenching buffer (1 M Tris-HCl [pH7.4]) and 40 μl of the reducing solution at room temperature for 30 min. The uncoupled fusion protein was removed by three washes with 5 ml of 1 M NaCl followed by 5 ml of degassed 0.05% sodium azide. The antibody was purified by incubation of 2 ml of anti-serum with the affinity column at room temperature for 1 hour. The unbound antibodies were removed with 20 ml of wash buffer (100 mM sodium phosphate, 150 mM NaCl, 0.1% Triton-100). The bound antibodies were eluted with 10 ml of elution buffer (150 mM glycine [pH2.5], 0.1% Triton-100, 150 mM NaCl), and 0.5 ml of fractions were collected and neutralized by the addition of 50 μl of 1.0 M Tris [pH9.5]. The fractions containing ≧0.05 mg/ml antibody were pooled and dialyzed against 2 liters of phosphate buffered saline at 4° C. overnight. The antibody solution was supplied with 0.05% sodium azide, aliquoted and stored at −80° C.

### Immunoprecipitation

Immunoprecipitation was performed as described [Wu et al., 1994, EMBO Journal, 13, 4823-4830], briefly, in vitro translated SPAF proteins (10 μl for ³⁵S-labeled proteins, 50 μl for unlabelled proteins) were incubated with 10 μl of anti-SPAF anti-sera or 1 μg of SpAb in 200 μl of lysis buffer (150 mM NaCl, 50 mM Tris [pH8.0], 1% NP-40, 1 mM phenymethylsulfonylfluoride, 50 μM aprotinin, and 10 μg/ml leupeptin) overnight at 4° C. The immunocomplexes were precipitated with 1.5 mg of protein-A-Sepharose CL-4B (a bead to which protein-A is immobilized) (Pharmacia, Piscataway, N.J.) for 2 hours at 4° C. The pellets were washed three times with lysis buffer, and fractionated through a 7.5% sodium dodecyl sulfate-polyacrylamide gel. For ³⁵S-labeled proteins, the gel was fixed, dried, and visualized by autoradiography. For unlabelled proteins, the gel was subjected to immunoblotting as described below.

### Immunoblotting

Mouse tissues were homogenized in dounce tissue grinders (Radnoti Glass, Arcadia, Calif.) in phosphate buffered saline and lysed in lysis buffer (150 mM NaCl, 50 mM Tris [pH8.0], 1% NP-40, 1 mM phenymethylsulfonylfluoride, 50 μM aprotinin, and 10 μg/ml leupeptin). For the preparation of cell lysate, 70% confluent cell cultures were lysed in the lysis buffer. Lysates were centrifuged for 15 min at 14,000 rpm. Twenty micrograms of lysate quantitated by Protein-assay (an acidic solution of Coomassie Brilliant Blue G-250 provided by Bio-Rad for protein quantitation) was incubated for 5 min at 100° C. in sample buffer (100 mM Tris Hcl [pH6.8], 20% glycerol, 2% sodium dodecyl sulfate and bromohpenoblue) containing 100 mM dithiothreitol and run on 7.5% sodium dodecyl sulfate-polyacrylamide minigel. After electroblotting onto nitrocellulose membrane (Schleicher and Schull, Keene, N.H.), blots were blocked overnight in TBST (50 mM Tris-HCl [pH7.5], 200 mM NaCl, 0.1% polyoxyethylenesorbitan) containing 5% nonfat milk powder, then incubated with SpAb (0.1 μg/ml) in TBST milk for one hour at room temperature. After washings in TBST, horseradish peroxidase-conjugated protein-A (Amersham) was added for one hour and immuno-complexes were visualized by chemiluminescence (Pierce).

### Immunochemical Staining

Five-micrometer paraffin sections of mouse tissues were depariffinized in xylene, and rehydrated through a graded series of ethenol concentrations. The sections were treated by microwave oven at medium setting for 10 min to unmask antigenic determinants, and permeabilized in 0.3% Triton-X 100/phosphate buffered saline for 10 min. The nonspecific binding of antibody was blocked with PAB (phosphate buffered saline, 0.1% sodium azide, 0.5% bovine serum albumin) containing 10% normal goat serum (Vector Laboratories, Burlingame, Calif.). The sections were then incubated with 0.1 μg/ml SpAb overnight at 4° C. After several washes in phosphate buffered saline containing 0.1% polyoxyethylenesorbitan, sections were incubated in biotin-labeled goat anti-rabbit IgG (Vector Laboratories), and reincubated in strepavidin-peroxidase conjugate (Research Genetics, Huntsville, Ala.). The color was developed by incubating the sections in diamino-benzidine tetrahydrochloride (DAB). Slides then were washed in H₂O and counterstained with hematoxylin, and mounted on coverslip using Permount (a histological mounting medium) (Fisher). Preimmune antibody at 1 μg/ml was used as a control for primary antibodies.

### Isolation and Molecular Cloning of SPAF

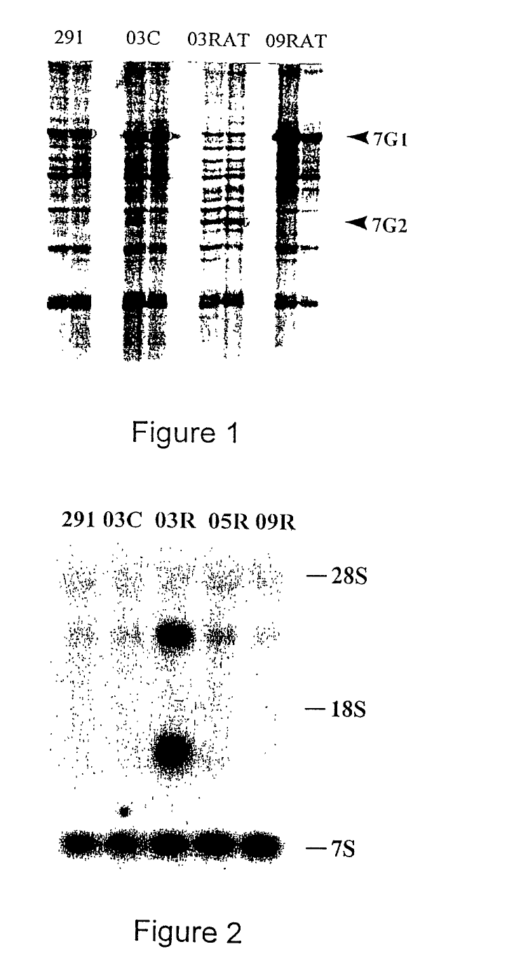

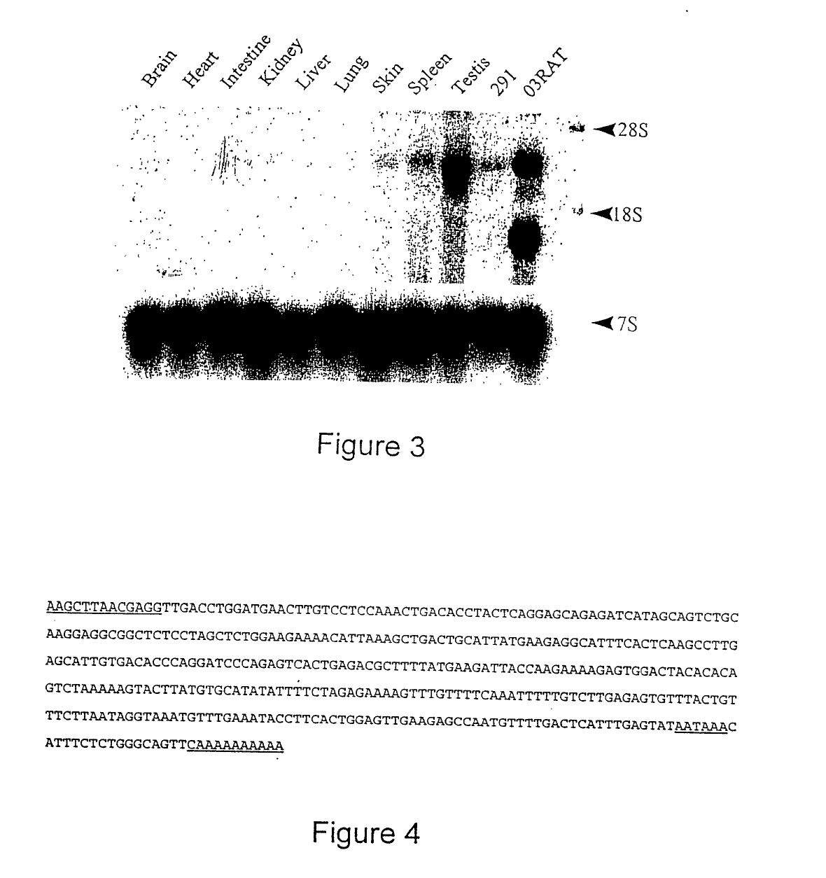

SPAF was initially identified as a cDNA tag termed 7G2 that appeared exclusively in the poorly differentiated squamous carcinoma by RNA differential display (FIG. 1). Northern analysis using this cDNA tag confirmed its expression pattern observed in the RNA differential display (FIG. 2). Two mRNA species corresponding to approximately 3 kb and 1.7 kb were detected exclusively in the poorly differentiated squamous cell carcinoma. Northern analysis of the tissue distribution of the transcripts showed that only testis had predominant expression of the 3 kb transcript (FIG. 3). The 1.7 kb mRNA was restricted to this poorly differentiated squamous cell carcinoma, suggesting that it may be a pathologically spliced variant or a truncated product due to a deletion within one allele of this gene. The sequence of this cDNA tag is 417 bp in length and contains flanking primer sequences identical to those used in the original display as underlined in FIG. 4. There is a putative polyadenylation signal immediate upstream of the poly A tail that is complementary to the anchored poly-deoxythymidine primer, suggesting that this cDNA fragment represents the 3′ end of the mRNA. A search of nucleotide and protein databases indicated that this was a novel gene product. Therefore, a 417 bp fragment of SPAF cDNA was used as a probe to screen a mouse testis cDNA library to isolate wild type full length SPAF. Three positive clones were obtained and the longest one was selected for further characterization. The 5′-end of SPAF was recovered by rapid amplification of cDNA ends (RACE) from mouse testis. The construction of the full length SPAF cDNA was achieved by taking advantage of the presence of a NotI site in the 5′ anchor primer and a unique BstEII site at position 186 of SPAF cDNA that overlaps sequences of RACE product and the cDNA isolated from the library.

### Production of Anti-SPAF Polyclonal Antibody (SpAb)

To determine the expression and localization of SPAF, the anti-SPAF polyclonal antibody SpAb was generated using the N-terminal portion of SPAF protein as immunogen. As discussed below, the central region of SPAF (from a.a. 350 to a.a 815) contains two ATPase modules which are highly conserved among proteins in the gene family to which SPAF belongs. Therefore the relatively unique N-terminal portion of SPAF (from a.a. 60 to a.a. 397) was selected and cloned in-frame with a poly-histidine tag under the control of the Lac Z promoter, a DNA sequence

| | |
|---|---|
| (TCGACTATAT CAGACTCTAT TTTTCCCAAT AAACGGCT CGTATAATGT | (SEQ. I.D. #10) |
| GTGGAATTGTGAGCGGTATA ACAATTTCAC ACAAGCT) | |

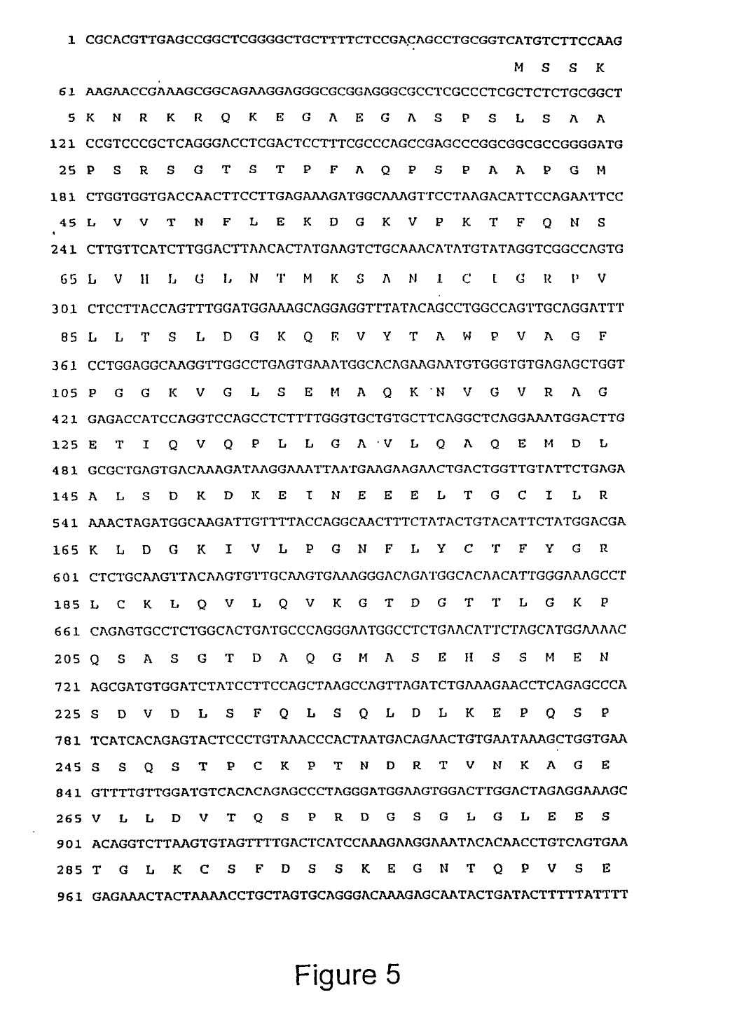

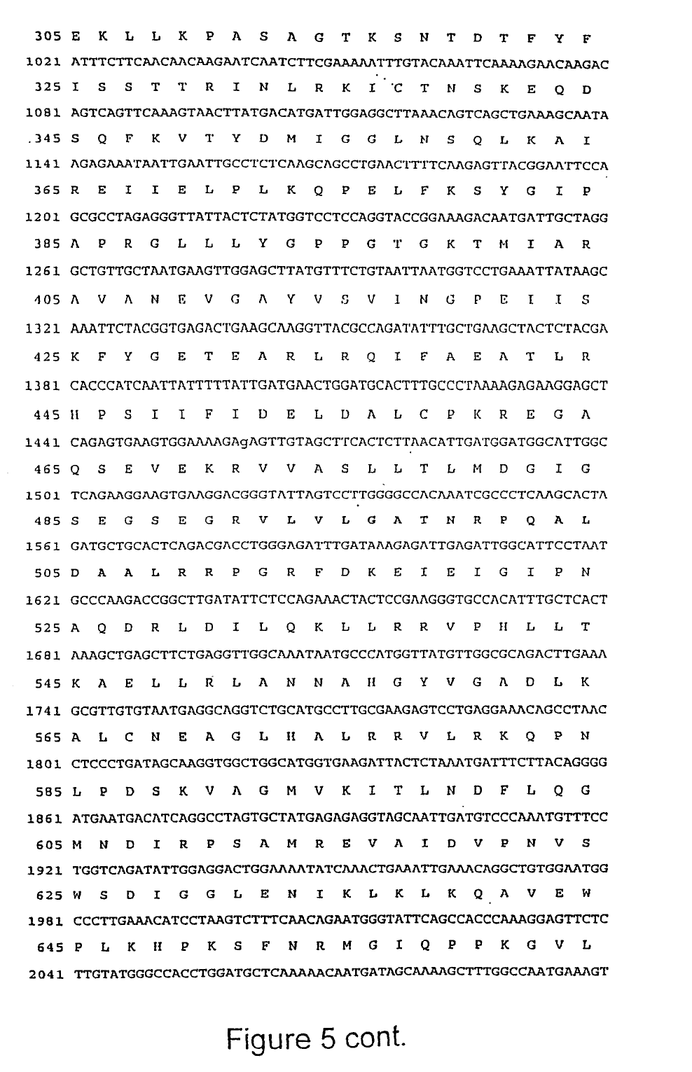

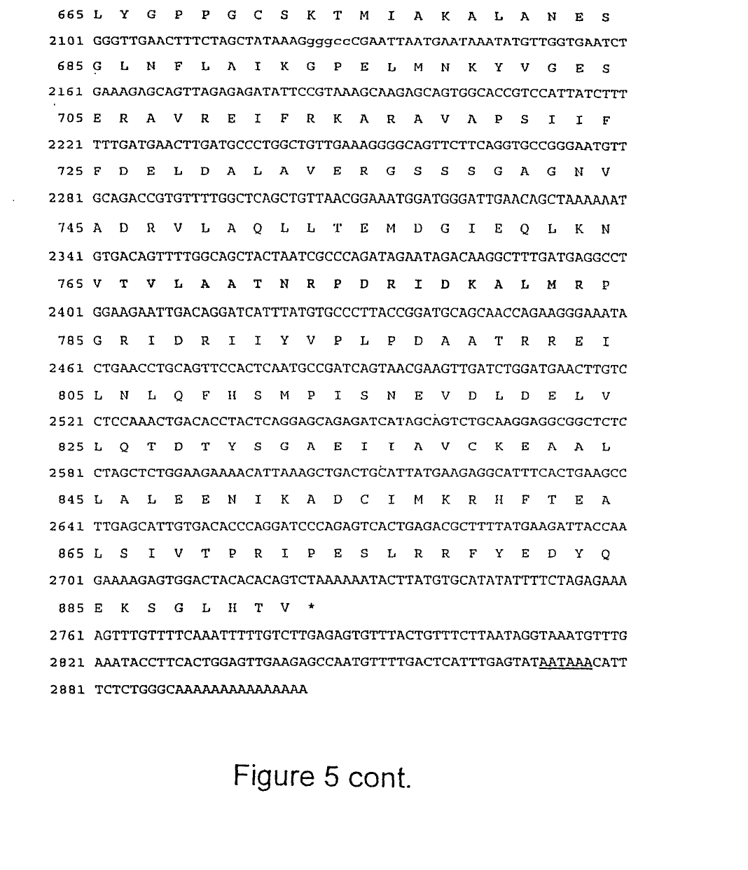

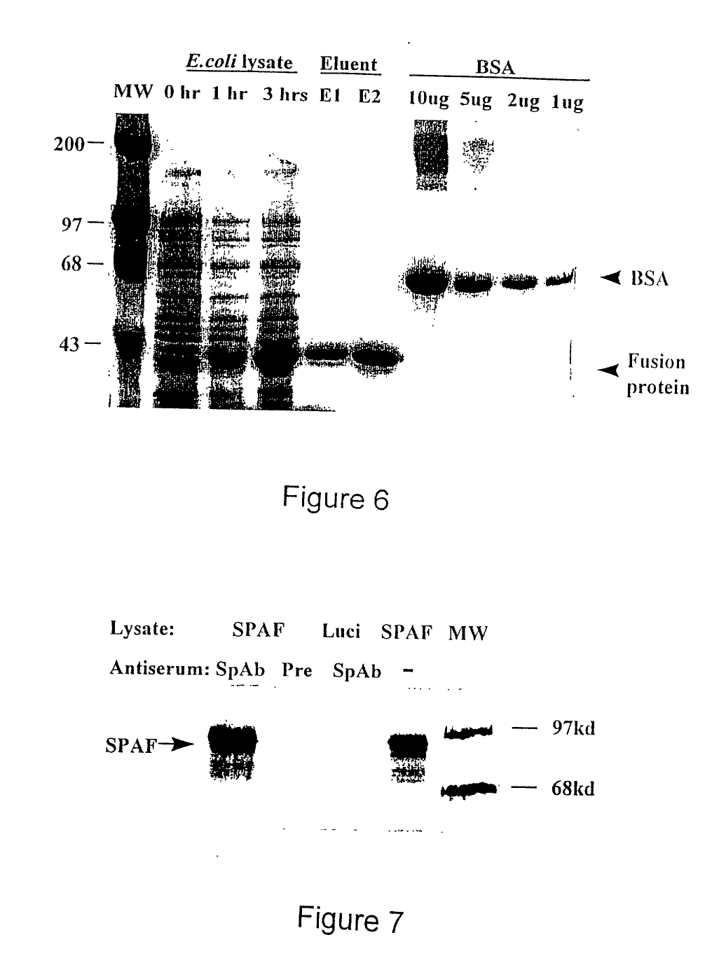

that activates the transcription of its downstream gene through the induction of galactose or its derivative isopropylthio-β-galactoside. The expression of the poly-histidine tagged fusion protein was induced by IPTG in log phase culture. Maximum expression was achieved after 3 hours induction (FIG. 5). The fusion protein produced in E.coli was purified by nickel affinity chromatography and used to immunize rabbits. The specificity of the affinity purified anti-SPAF antibody, SpAb, was tested by immunoprecipitation of in vitro translated SPAF protein as shown in FIG. 6.

### Sequence Analysis of SPAF cDNA

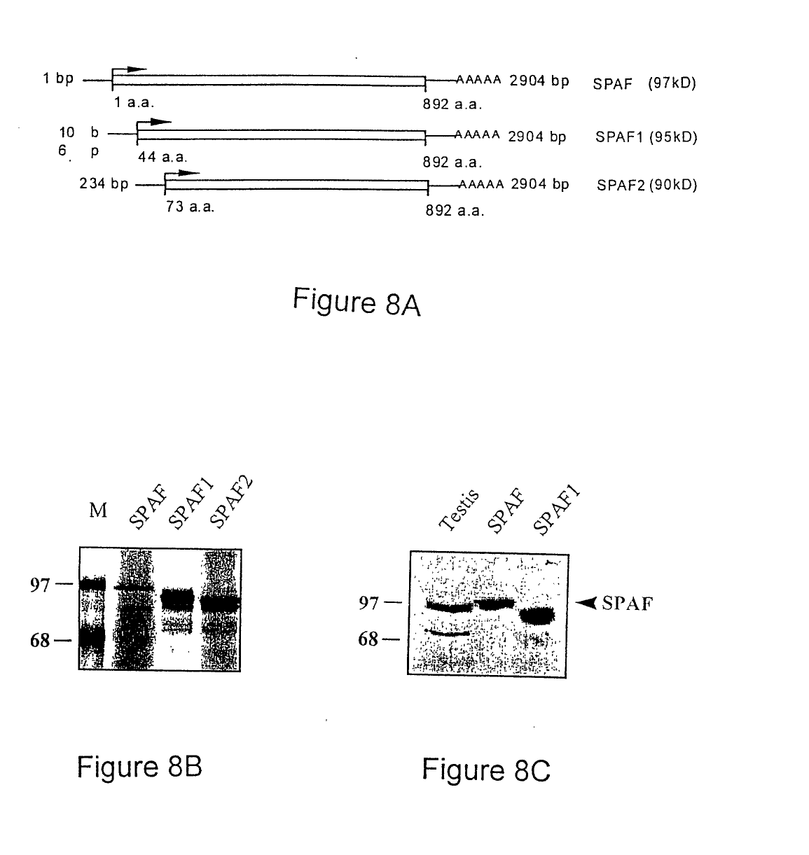

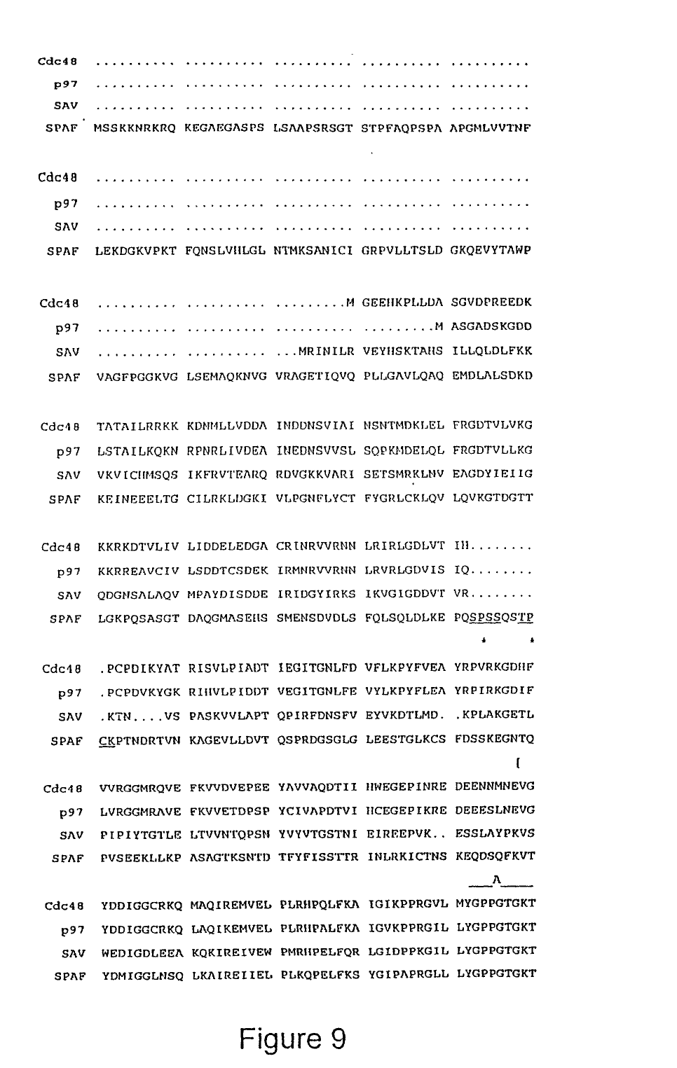

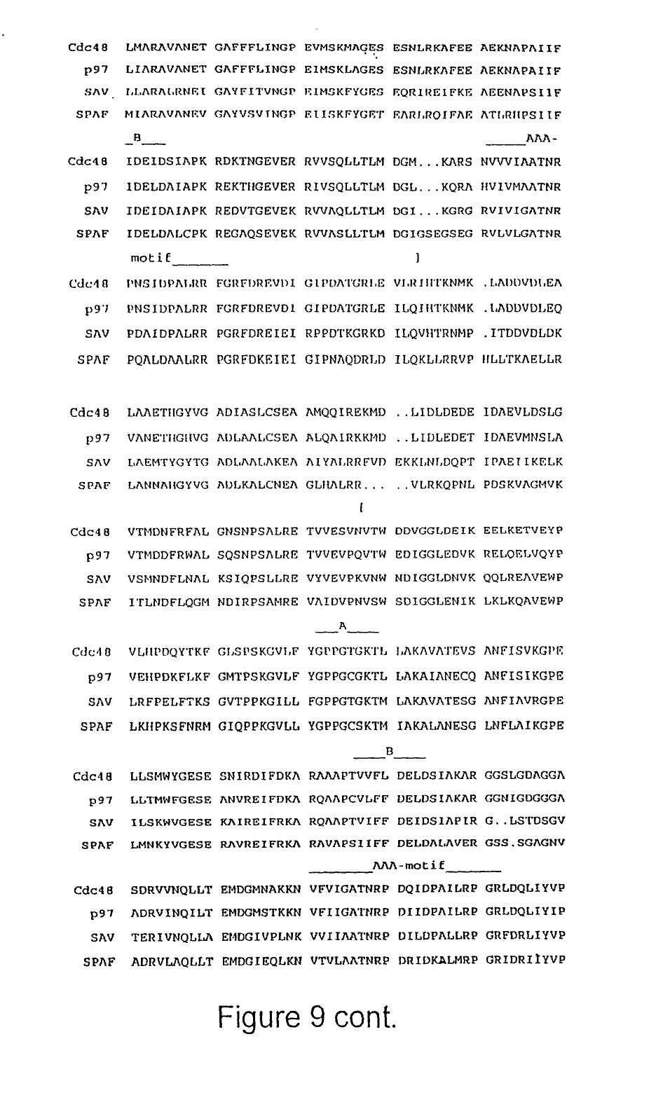

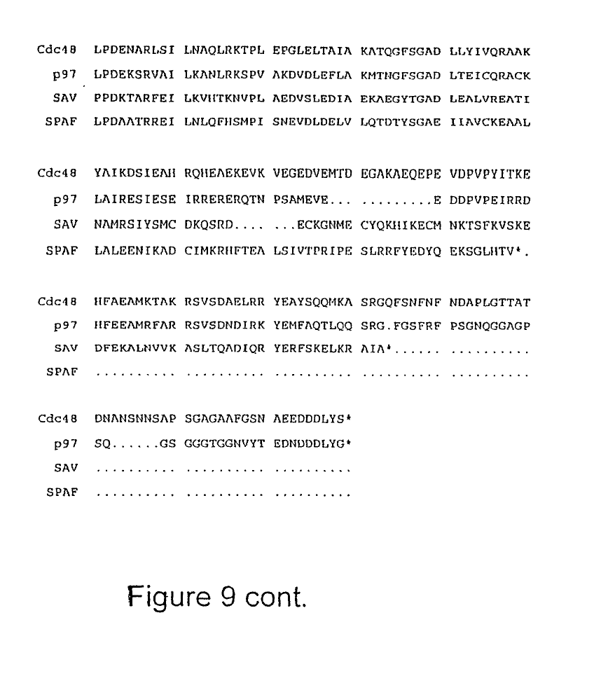

The SPAF cDNA constructed from the cDNA library clone (106-2904) plus RACE PCR product (1-106) totaled 2904 bp. It contained a long open reading frame between nucleotide position 49 and 2725 which encodes a polypeptide of 892 amino acids (FIG. 7). To confirm the validity of the predicted open reading frame, the full length SPAF cDNA was expressed by in vitro transcription/translation in the presence of [³⁵S] methionine and subjected to SDS polyacrylamide gel electrophoresis. A product of 97 kDa was detected as expected by calculation from the deduced amino acid sequence of SPAF. Since the first ATG codon is not surrounded by a translation start site consensus sequence [Kozak, 1991, Journal of Cell. Biology, 115, 887-903], the authentic initiation codon of SPAF was examined. Two 5′-truncated forms of SPAF, SPAF1 and SPAF2, were made to eliminate the first and the second ATG codon, yielding expected polypeptides of 92 kD and 90 kD respectively (FIG. 8A). If the downstream ATG was the initiation codon of SPAF, the polypeptide generated from the full length SPAF cDNA would migrate at the same position of the truncated form. FIG. 8B shows that the molecular weight of the truncated forms decreases in proportion to the size of the deletion, demonstrating that the first ATG is the initiation codon of SPAF cDNA. As no termination codon in frame was found in the 5′ region upstream of the presumed initiation codon, the full length open reading frame was confirmed by comparing the sizes of in vitro translated SPAF proteins with endogenous SPAF protein by immunoblotting analysis. Because the amounts of in vitro translated proteins were too low to be detected by immunoblotting directly, in vitro translated SPAF proteins were enriched by immunoprecipitation before immunoblotting analysis. As shown in FIG. 8C, the protein translated from the full length SPAF cDNA migrated at the same position of its endogenous counterpart. High GC content (69%) was found in the 5′-untranslated region, which may be an impediment to translation as indicated by increased translation efficiency of 5′-truncation mutants. A typical polyadenylation signal, AAUAAA, is found immediately upstream of the poly (A) tail. The 3′-untranslated region is relatively high in AU content (70%) and contains four AUUU destabilizing elements [Shaw et al., Cell, 46, 659-667, 1986]. These features suggest that the expression of SPAF is highly regulated both at the post-transcriptional and translational levels. A database search using BLAST (National Center for Biotechnology Information) revealed that SPAF is a new member of the AAA-protein family (ATPase Associated with diverse cellular Activities). A duplicated nucleotide binding domain of approximately 200 amino acids containing an AAA-protein family signature (VxVLxATNx[4]IDxALxR) [Kunau et al., Biochemie, 75, 209-224, 1993] and the Walker A and B nucleotide binding motifs [Walker et al., EMBO Journal, 1, 945-951, 1982] was found in a region from 350a.a. to 815a.a of SPAF protein (FIG. 7). The sequence similarities between SPAF and other members containing two ATPase modules range from 50% to 66% (identities 25%-45%) over the whole length of the proteins (Table 1), with the ATPase modules being highly conserved. Both N-terminal and C-terminal regions of SPAF show no significant similarities with corresponding regions of other members in this family (FIG. 9). Several unique features have been found in the N-terminal region of SPAF. Firstly, the N-terminal region is about 130 a.a. longer than that of other closely related proteins. Secondly, the extreme N-terminus of SPAF is rich in positively charged arginine and lysine and hydroxylated serine which implies a putative mitochondrial matrix-targeting sequence. Thirdly, two putative cdc2 phosphorylation sites [Nigg, Seminars in Cell Biology, 2, 261-270, 1991] lie side by side immediately upstream of the first ATPase module. No DDDLY motif (a polypeptide motif present in some AAA-proteins. D: aspartic acid; L: leucine; Y: tyrosine) is found in the C-terminal region of SPAF. This motif is associated with a ring-like structure found in the VCP (valosine-containing protein), p97, and CDC48 (a yeast homologue of SPAF) and many other heat shock proteins [Mian, Trends Biochem-Sci. 18, 125-127, 1993], suggesting that SPAF has a different quaternary structure.

### Expression of SPAF in Normal Testis

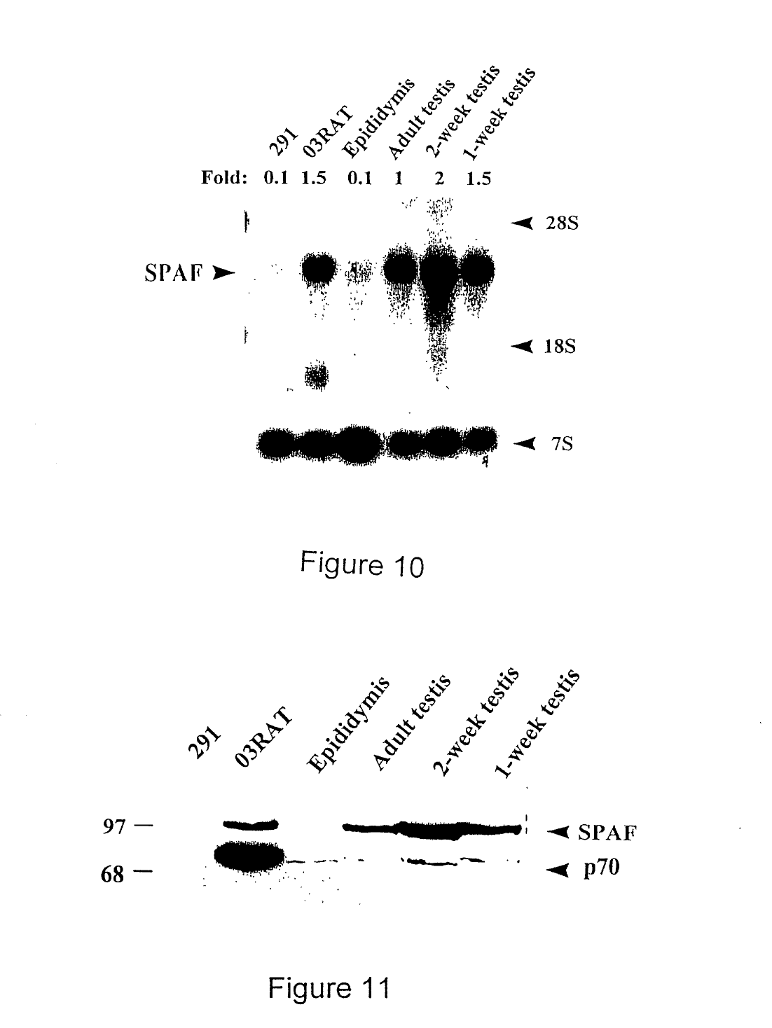

The testis is composed mainly of germ cells of three categories: spermatogonia, spermatocytes, and spermatids, which represent different phases of spermatogenesis [Clermont, Physiological Reviews, 52, 198-236, 1972]. In mice, spermatogenesis begins shortly after birth, and the composition of the seminiferous epithelium undergoes predictable changes through development [Bellve et al., J. of Cell Biol., 74, 68-85, 1977]. On postnatal day 8, the seminiferous tubule contains 27% spermatogonia and 73% Sertoli cells; on day 14, it contains 12% spermatogonia, 51% spermatocytes (which include cells up to pachytene stage), and 37% Sertoli cells; in the adult, it contains 4% spermatogonia, 22% spermatocytes, 71% spermatids, and 3% Sertoli cells. To examine which testicular cells express SPAF, we analyzed the expression of SPAF in adult epididymis and testes from prepuberal and adult mice. As shown in FIG. 10, high levels of SPAF mRNA were detected in all RNA samples from testes but not from epididymis. Densitometric analysis showed 1.5 fold and 2 fold higher SPAF RNA steady state levels in 1-week and 2-week old testes as compared to adult testis. The peak SPAF expression is proportional to the maximal percentages of spermatogonia and spermatocytes during development, indicating that the expression of SPAF is correlated with spermatogonia and spermatocytes but Sertoli cells or spermatids.

In general, the regulation of transcription is the major control of gene expression, and the presence of mRNA often indicates the expression of protein. However, it has been observed that transcription and translation are uncoupled for some genes during spermatogenesis [Braun et al., Genes and Devt., 3, 793-802, 1989]. Therefore, the expression pattern of SPAF in spermatogenic cells was analyzed by Western immunoblotting. SPAF protein was detected by reactivity with SpAb in both prepuberal and adult testes, both of which contain cells at early stages in spermatogenesis, but not in epididymis, which contains differentiated spermatids (FIG. 11). The highest expression of SPAF was observed in prepuberal testes, consistent with the northern analysis data. It is of interest that two bands were detected by immunoblotting in the testes and epidermal cells, one of 97 kD and one of 70 kD. The 97 kD band co-migrated with the in vitro translated SPAF protein, indicating that it is the cellular SPAF protein.

### Localization of SPAF Protein

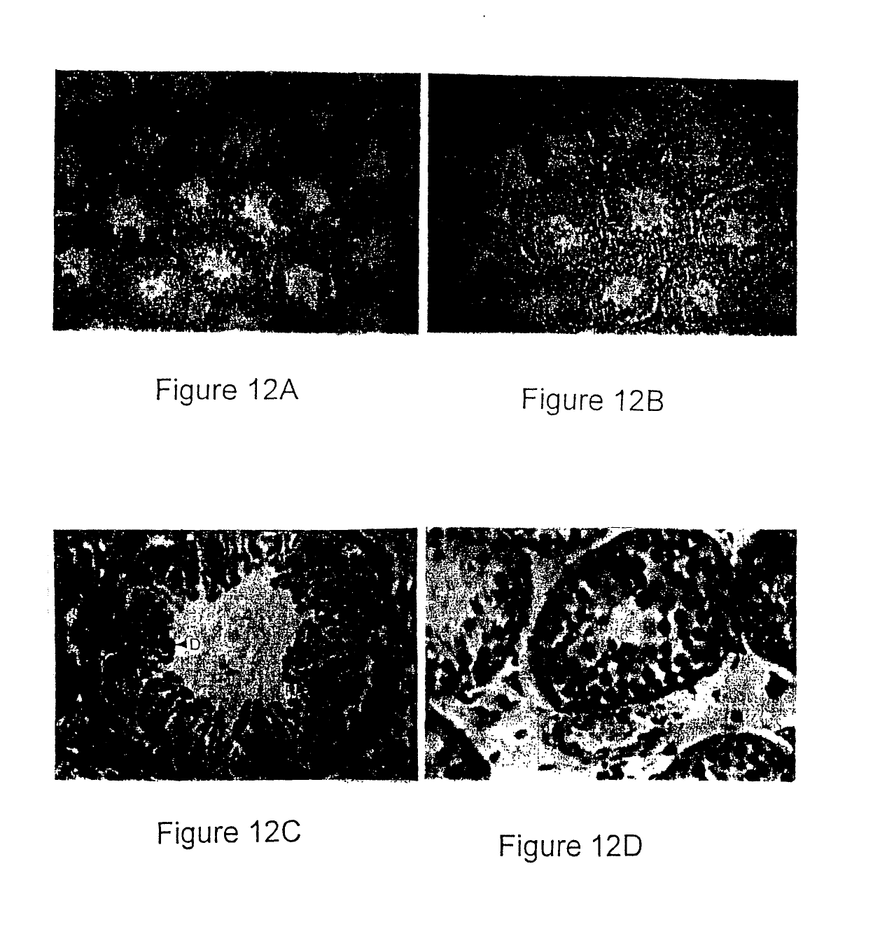

In order to verify the expression of SPAF in spermatogenic cells and its subcellular location, immunostaining was performed with sections from prepuberal and adult testes (FIG. 12). The SPAF protein was found in basal cells of the seminiferous tubule, that is in spermatogonia and in spermatocytes at lepotene, zygotene, and early pachytene stages. The level of SPAF protein dramatically decreased in primary spermatocytes at the late pachytene stage and was absent in the spermatogenic cells thereafter. No staining was observed in other cells of testis including Sertoli cells, interstitial cells, and mature sperm in epididymis. All the spermatogenic cells were positively stained in two day old testis, which was consistent with the result from adult testis since germ cells develop up to pachytene stage at postnatal day 14. The intracellular staining pattern indicated that SPAF protein was localized in the cytoplasm rather than nuclear.

### Immunoblotting Analysis of SPAF Expression During Tumorigenesis

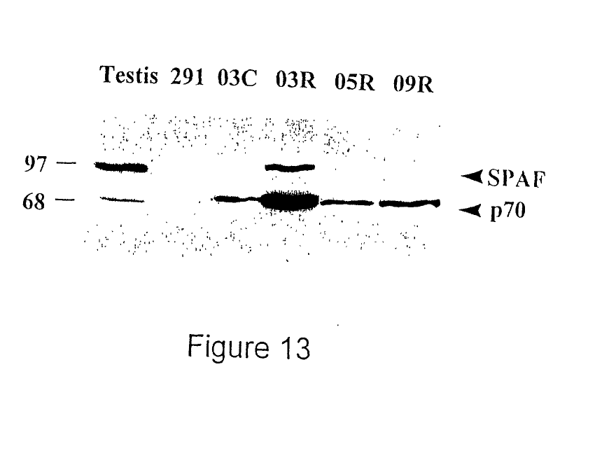

Immunoblotting analysis of SPAF expression in the cloned epidermal cell of the model system was performed using SpAb. As shown in FIG. 13, the 97 kD protein band was detected exclusively in the 03RAT cells but also the other tumor derivatives and the initiated cell, except the normal progenitor cell 291, which was negative or very weakly positive. Possible explanations for this smaller protein are: 1) it is a degraded form of SPAF; 2) it is an unrelated protein that shares epitopes recognized by this antibody. It seems more likely to be an unrelated protein since Northern analysis of the clonal epidermal cell model (FIG. 2) showed no detectable SPAF mRNA in cells with the smaller protein only.

In search of altered gene expression during multistage carcinogenesis, a novel AAA-protein, SPAF, has now been purified and identified as a normal spermatogenesis associated factor aberrantly expressed at the malignant conversion stage in a cloned epidermal cell model of carcinogenesis. In the adult male mammal, spermatogonia and primary spermatocytes are the only source of potential totipotent diploid cells that provide a genetic contribution to the next generation. Retention of a primitive phenotype is one of the characteristics of tumor cells. There is precedent for aberrant expression of a germ cell gene contributing to malignant transformation. Ectopic expression of mos, a germ cell-specific gene, contributes to genetic instability in transformed cells, probably by mediating a meiosis-like phenotype in somatic cells. Overexpression of SPAF may contribute to malignant transformation of cell precursors by imparting a characteristic of the germ cell phenotype. Interference with this activity of SPAF in tumor cells may be developed as an approach to reverse the malignant phenotype. One such approach would make use of the highly conserved and well characterized ATPase modules of SPAF. The ATPase module is approximately 200 amino acids and is composed of consensus sequences for ATP binding and hydrolysis, the Walker type-A and -B motifs. The Walker A consensus region is believed to form the floor of the nucleotide-binding pocket, and the Walker B region is thought to form a loop that overlays the pocket and positions Mg²⁺ ions [Koonin, J. of Mol. Biol., 229, 1165-1174, 1993]. These regions lend themselves to being targeted by small molecules. The unique N-terminal and C-terminal domains of SPAF, on the other hand, can be utilized as tumor-specific markers in developing antibodies for diagnosis and targeted cancer therapy. A prominent advantage to SPAF as a target in cancer therapy is that side effects of the therapy would be restricted in germ cell only. There are potential applications of the SPAF gene and its derivatives outside of the cancer field. SPAF has potential application in human fertility as indicated by its unique expression in spermatogenesis. In addition, SPAF may prove to be an early gene marker of embryonic stem cells in spermatogenesis. The molecular probes and antibodies declared here enable the determination of the potential of this gene and its products and derivatives for these applications in human cells.

SPAF DNA (SPAF), SPAF cDNA, SPAF proteins, SPAF antibodies and anti-SPAF antibodies are intended to include human forms obtained by procedures similar to those described above.

Various bases in SPAF DNA, cDNA and amino acid residues in SPAF proteins, antibodies and anti-antibodies may be altered while maintaining SPAF DNA, cDNA protein and antibody specificity function. Such tolerated alterations are readily determined by making a substitution and using the substituted form in place of the unsubstituted form and comparing results.

The terms SPAF, SPAF cDNA, and anti-SPAF antibodies as used herein are intended to include tolerated alterations as described above. The above terms are also intended to include marker, and tumor inhibiting conjugates of SPAF, SPAF cDNA, and anti-SPAF antibodies.

SPAF gene in pBluescript in E. coli HB101 is being deposited with ATCC.

### Presence of SPAF in Humans

Using mouse SPAF cDNA, as above described, a 472 bp EST human homologue sequence was found at GenBank accession number A1791698. The sequence was identified from a human kidney tumor.

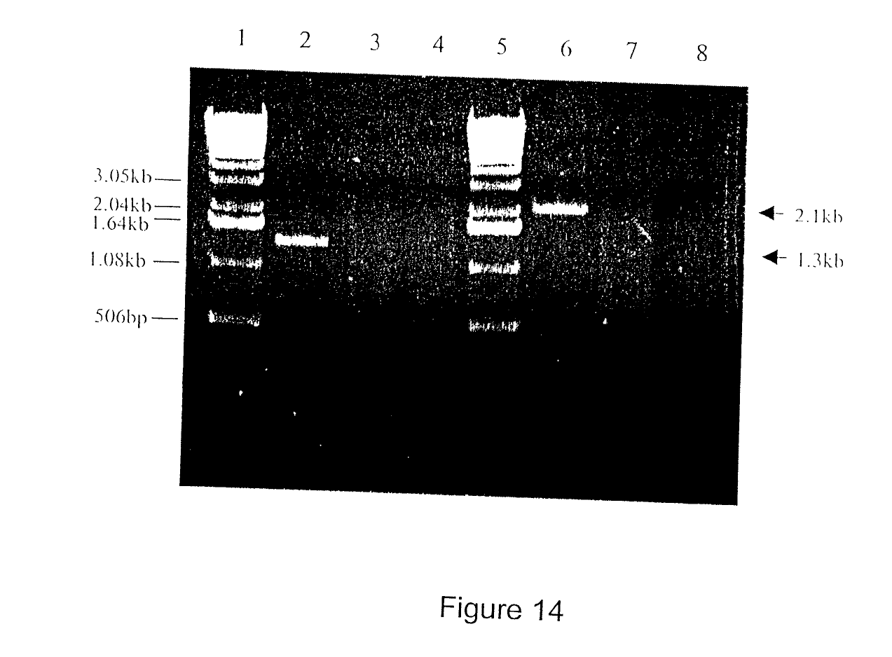

The full length (5′ end to 3′ end) of the human SPAF (hSPAF) cDNA was cloned by RACE-PCR (Rapid Amplification of cDNA Ends). Total RNA was isolated from human embryo kidney 293 cell (ATCC No. CRL-1573) and subjected to reverse transcription with oligo dT (Clontech) as a primer. The 5′ cDNA end of the hSPAF was amplified by a conventional universal primer mix (UPM, Clontech) and an oligonucleotide (H3-3) derived from nucleotide 125 to 145 of human EST clone (GenBank accession #AI79698). The PCR products were analyzed by electrophoresis on 1% agarose gel and visualized by Ethidium Bromide staining. The results are shown in FIG. 14. Lane 2 of the gel is the 1.3 kb 5′ RACE product amplified by UPM and human SPAF specific primer #3-3. Lane 6 is the 2.1 kb 3′ RACE fragment amplified by UPM and the gene specific primer H2-5. Control amplifications by UPM alone are shown in lanes 3 and 7 for 5′ RACE-PCR and 3′ RACE-PCR respectively. Lanes 4 and 8 are the control PCR reactions by H3-3 and H2-5 respectively. Lanes 1 and 5 are 1 kb DNA ladders. The sizes of the PCR products are as shown in FIG. 14.

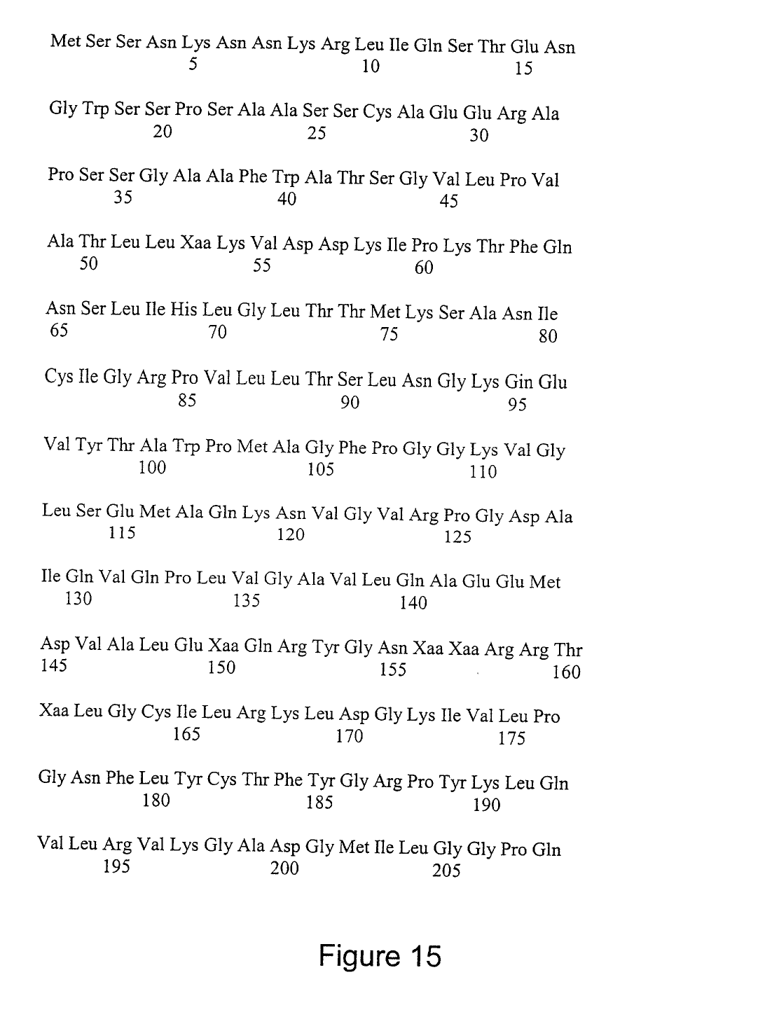

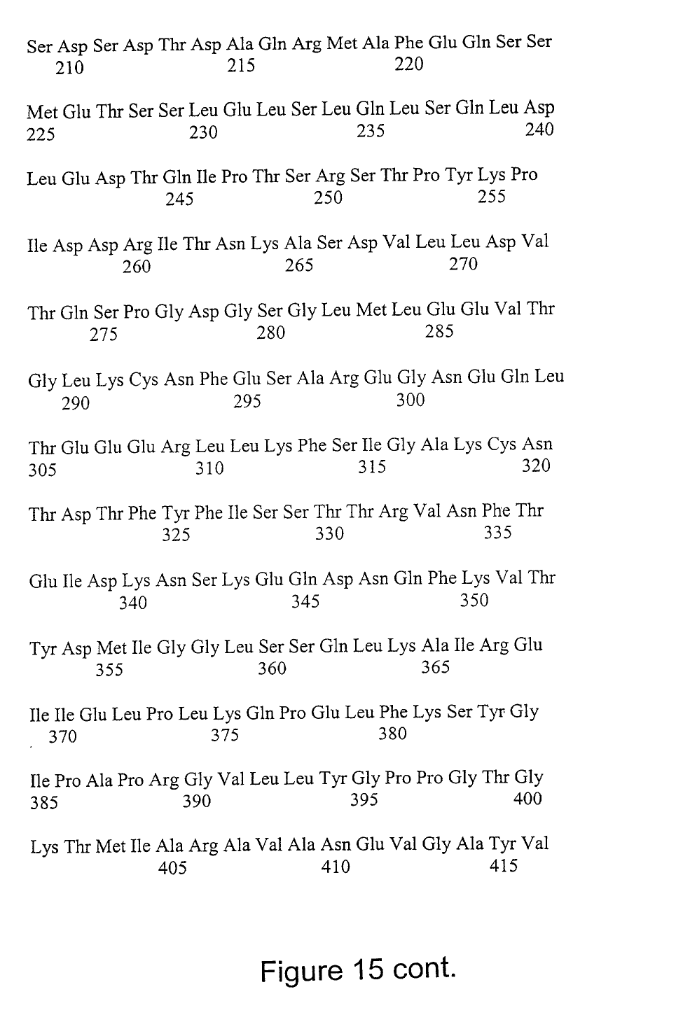

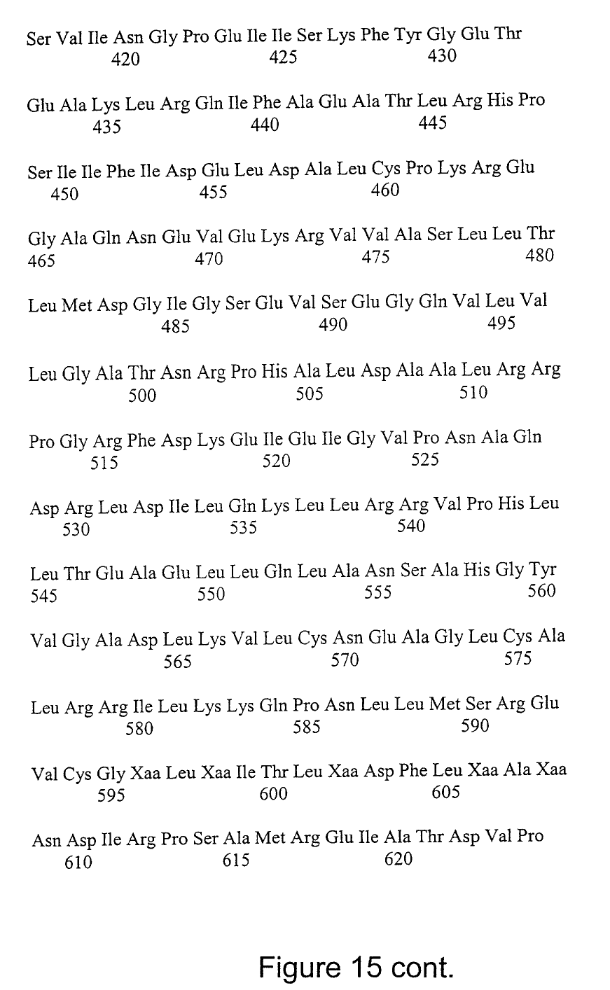

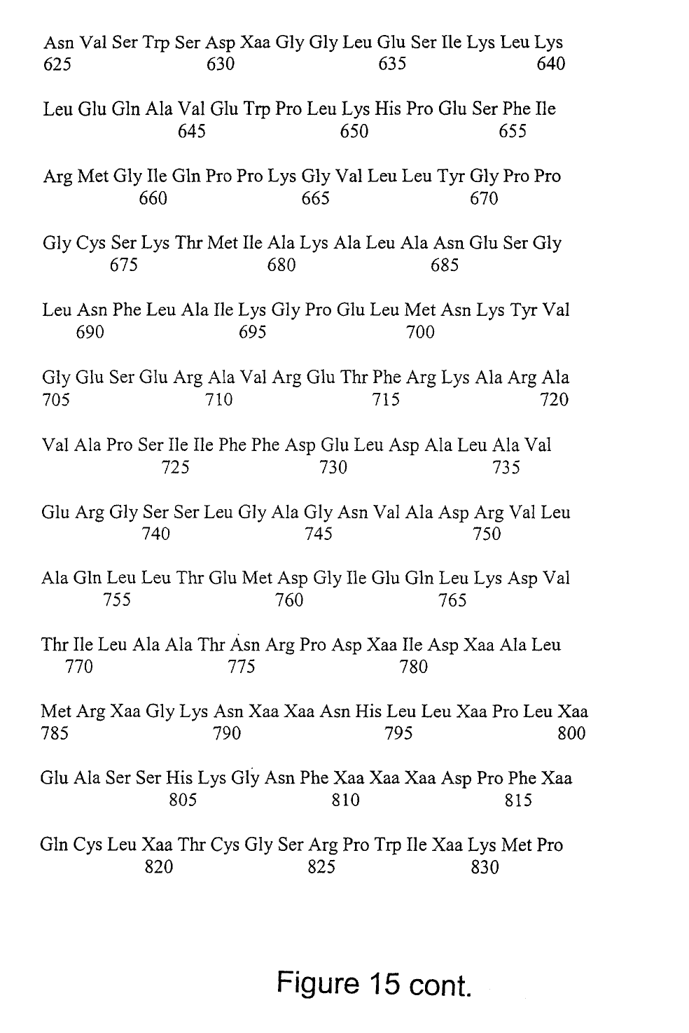

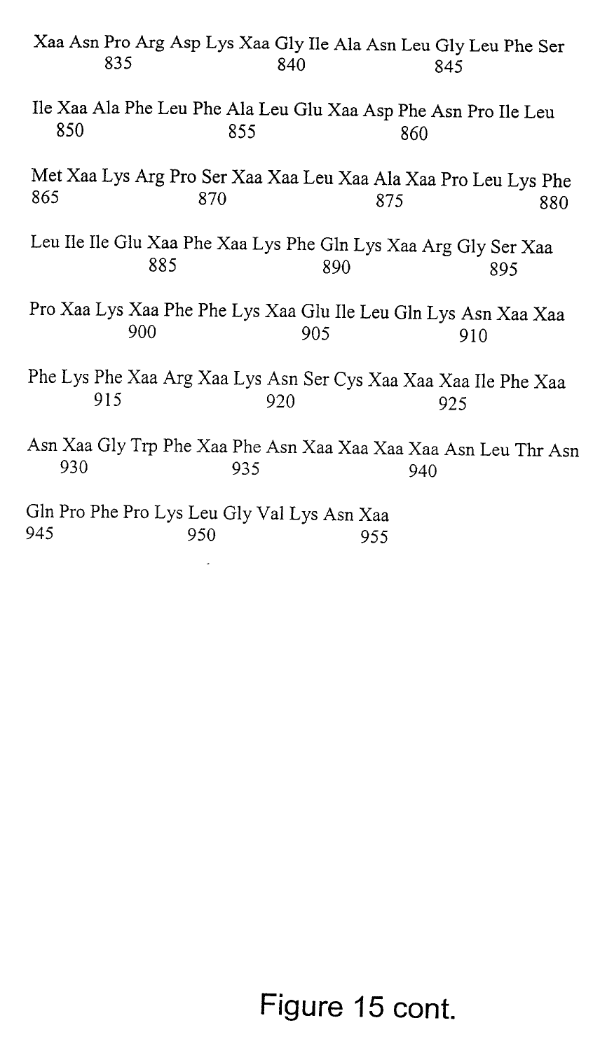

Protein corresponding to the human SPAF codon has been preliminarily sequenced. The results are shown in FIG. 15. Walker A and Walker B motifs are underlined. The AAA-protein family signature sequence is italicized and underlined.

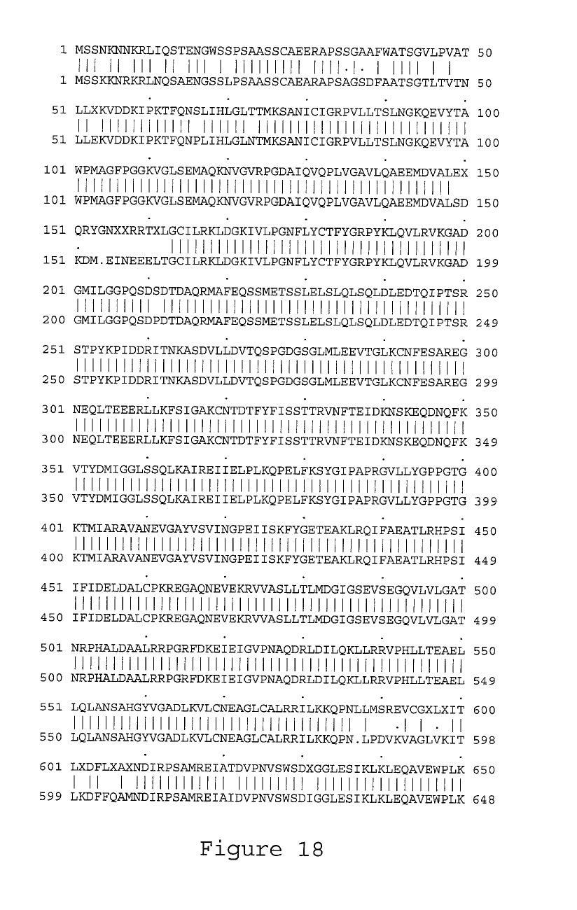

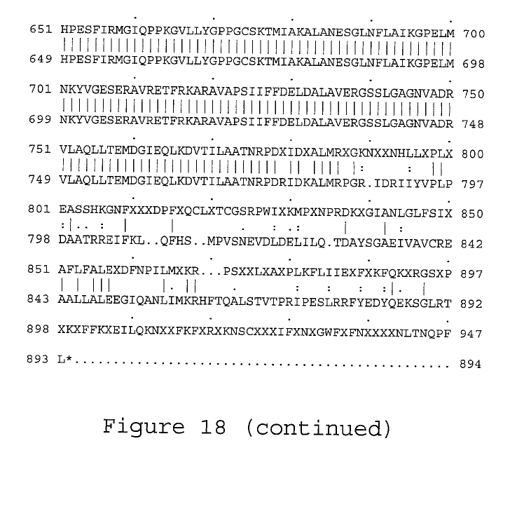

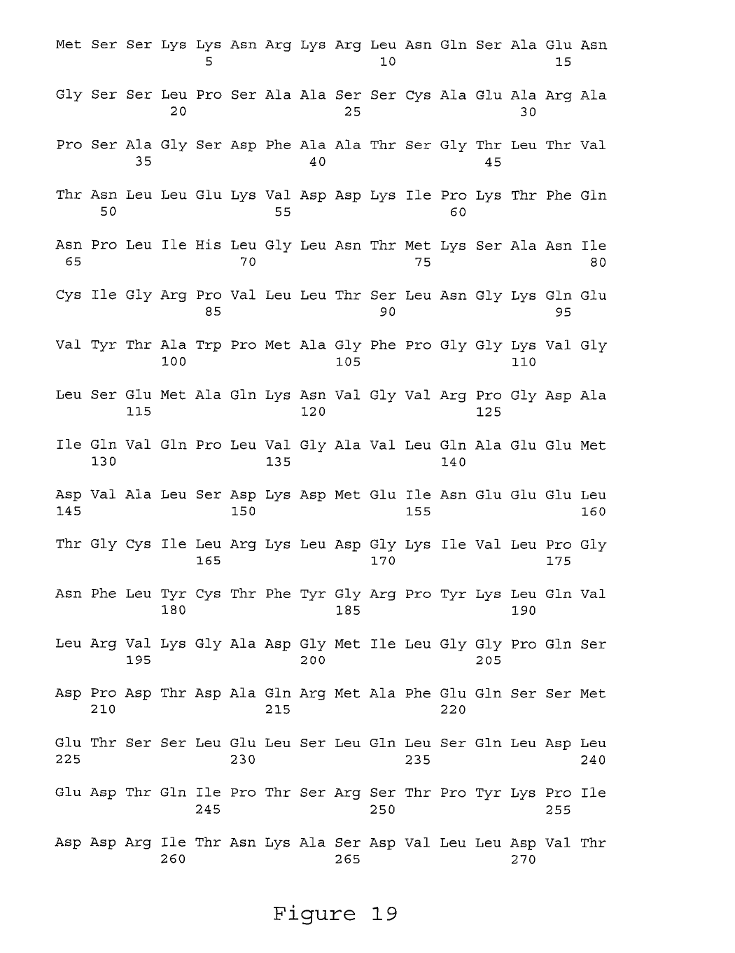

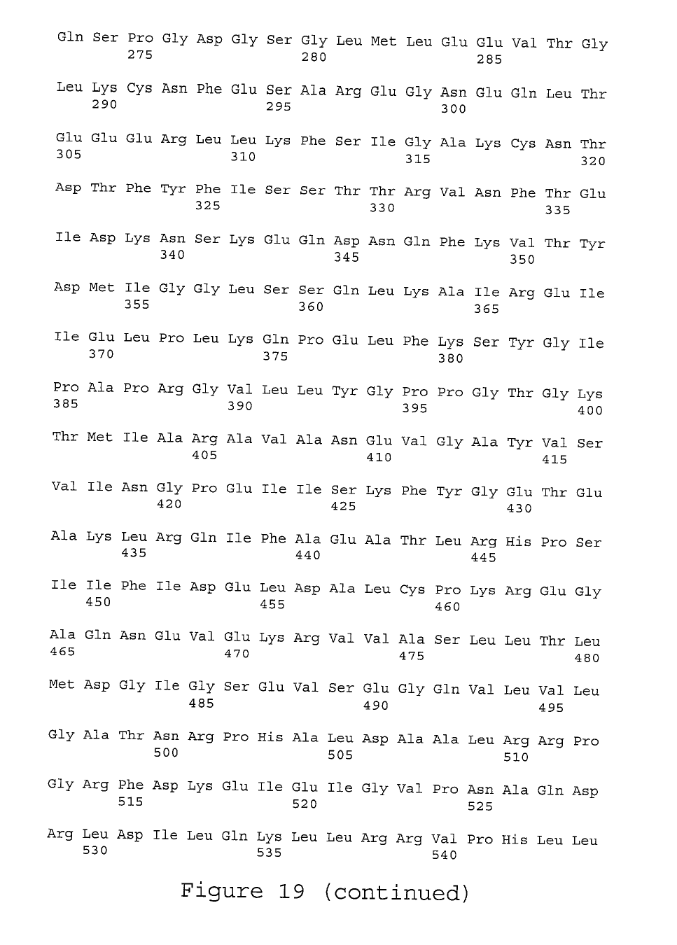

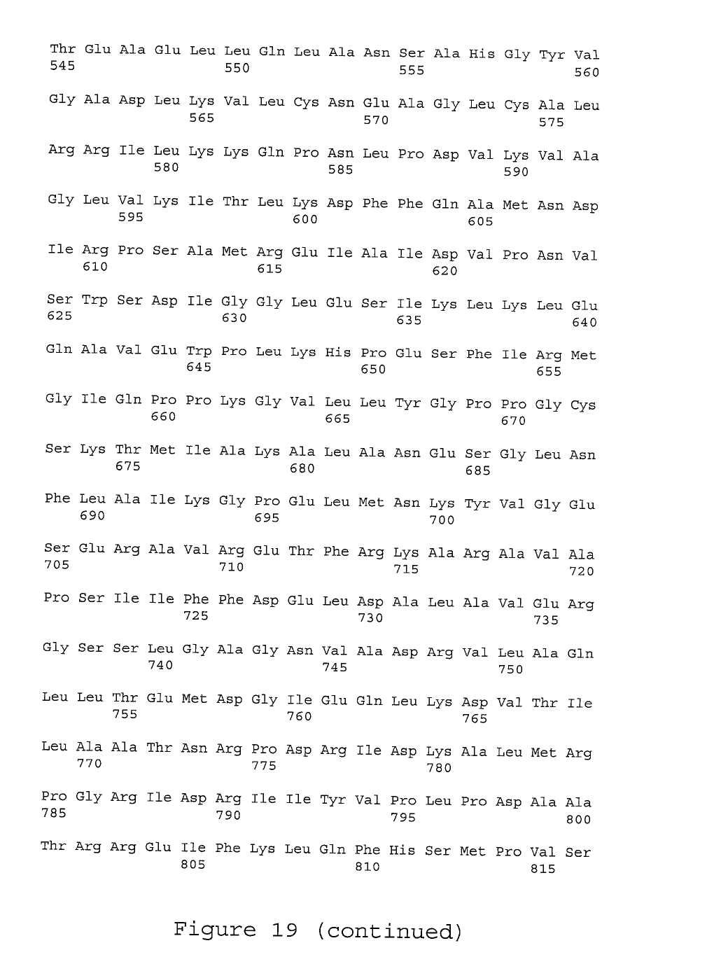

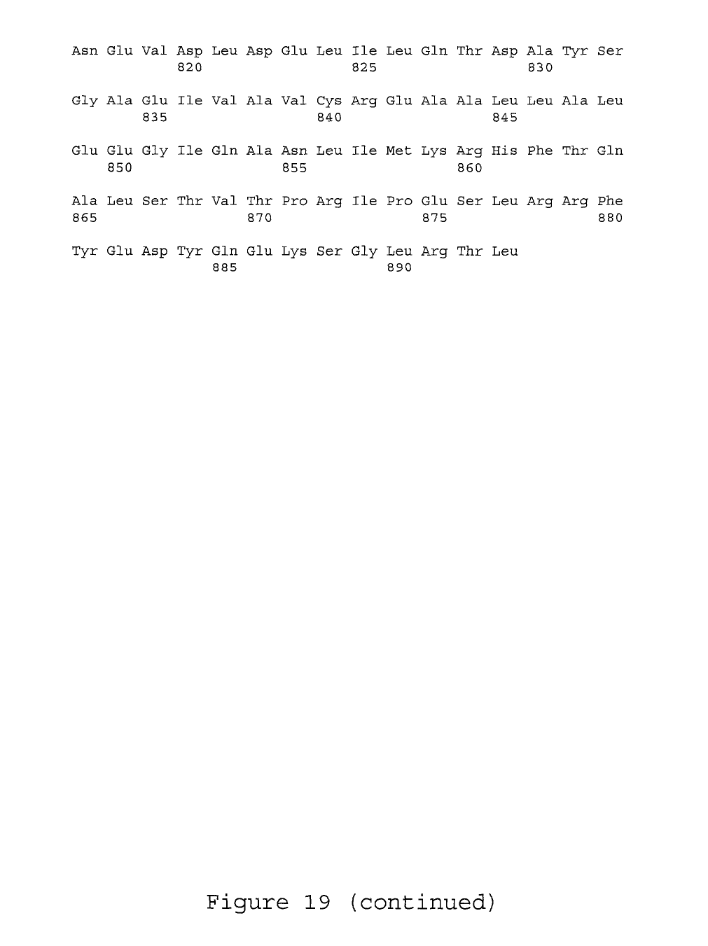

A further checked and deduced sequence for human SPAF protein is shown in FIG. 19. A comparison between the preliminary human SPAF protein sequence and the further checked and deduced human SPAF protein is shown in FIG. 18.

Sequence analysis using the unambiguous 61 to 590 portion of hSPAF sequence (total of 1587 base pairs in length) showed the expected 40-58% identity with other AAA-protein family members of human and other species and a definitive 82% identity with mouse SPAF as shown in Table 4.

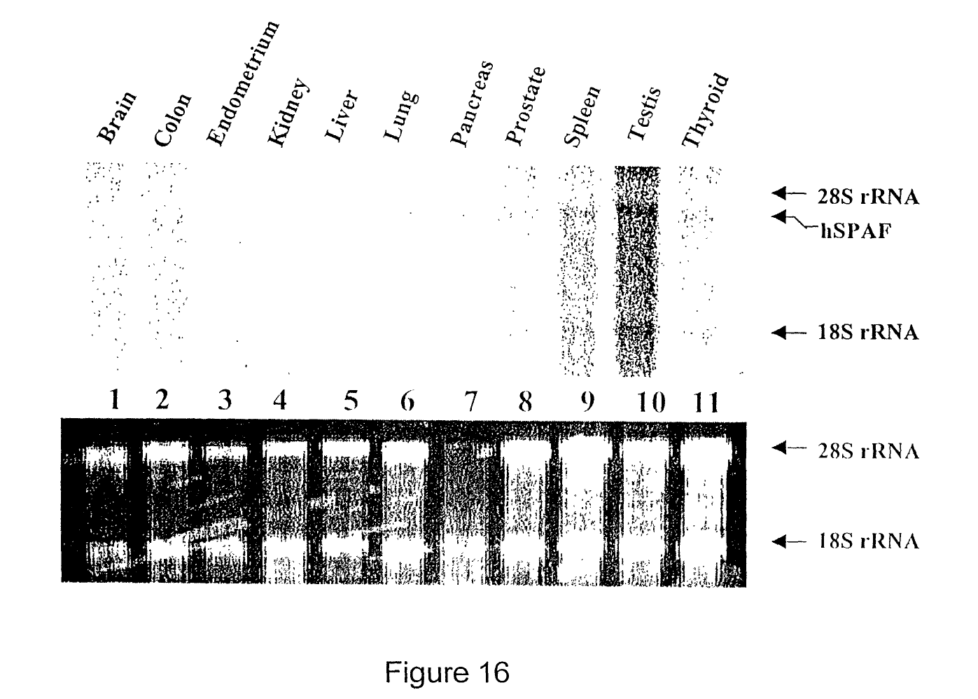

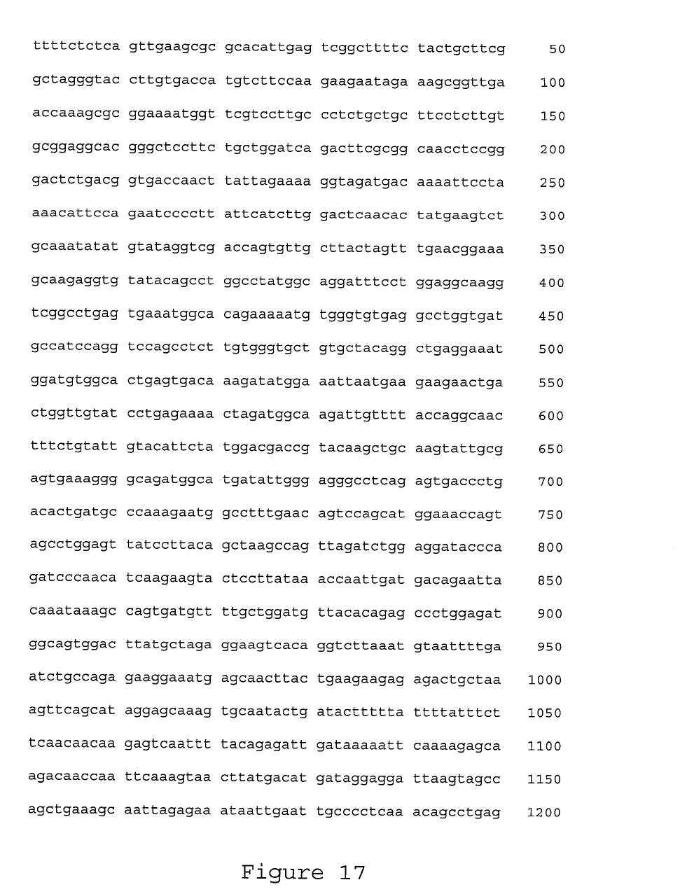

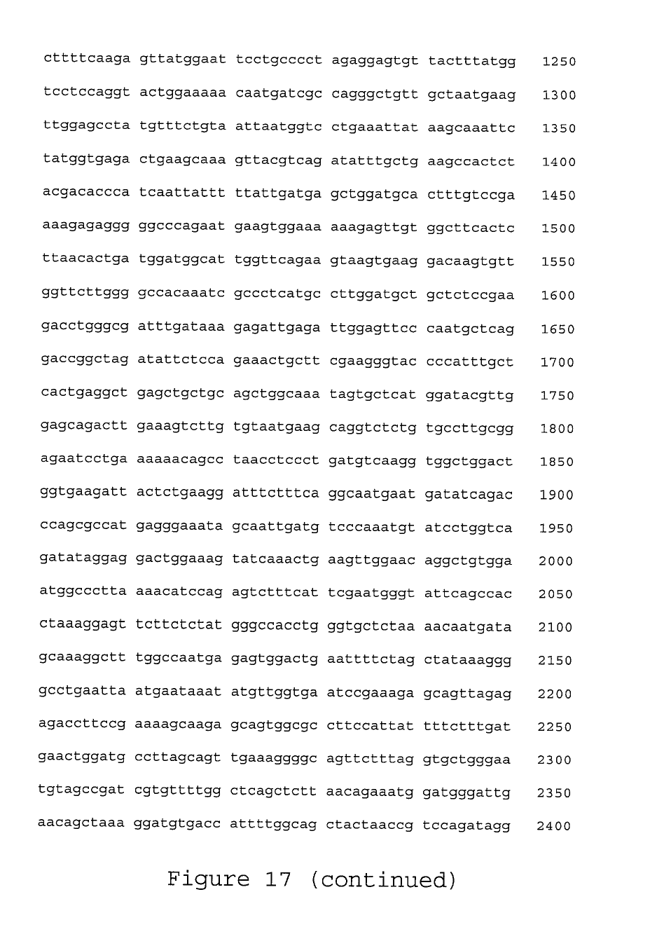

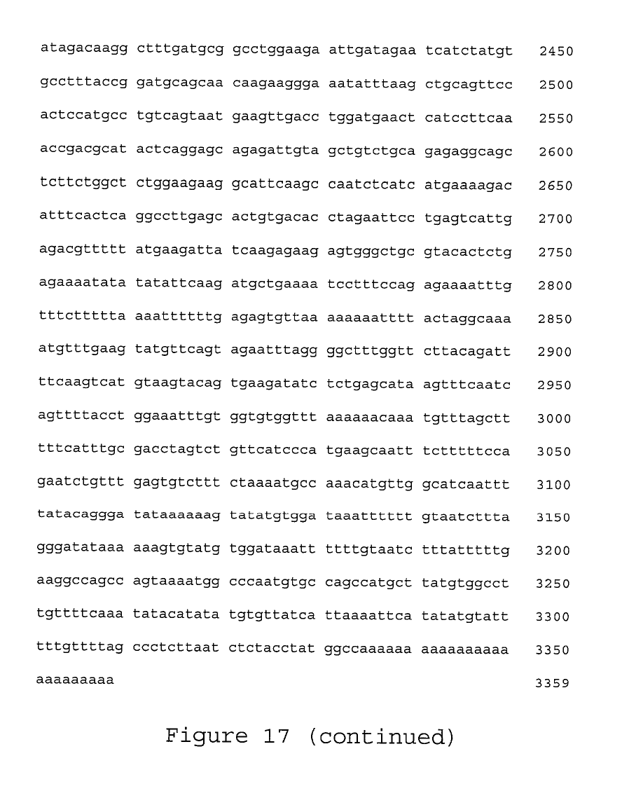

Northern blotting of human SPAF gene expression pattern in normal human tissues showed predominant expression in testes, consistent with the observation for mouse SPAF. Expression profiles of human SPAF in normal tissues are shown in FIG. 16.

Total RNA from the normal human tissues indicated were extracted using Trizol (Gibco BRL) and 10 μg of each was separated on a 1.2% agarose formaldehyde gel. The RNA's were transferred and immobilized onto a nitrocellulose membrane and hybridized with the 1.3 kb fragment derived from the 5′ end of the human SPAF gene (FIG. 14). The band representing human SPAF transcripts is indicated. Ethidium Bromide staining gel is shown as a loading control on the lower panel of FIG. 16.

SPAF gene sequence uncovers a bidirectional gene promoter structure with reciprocal control of a human growth factor pathway.

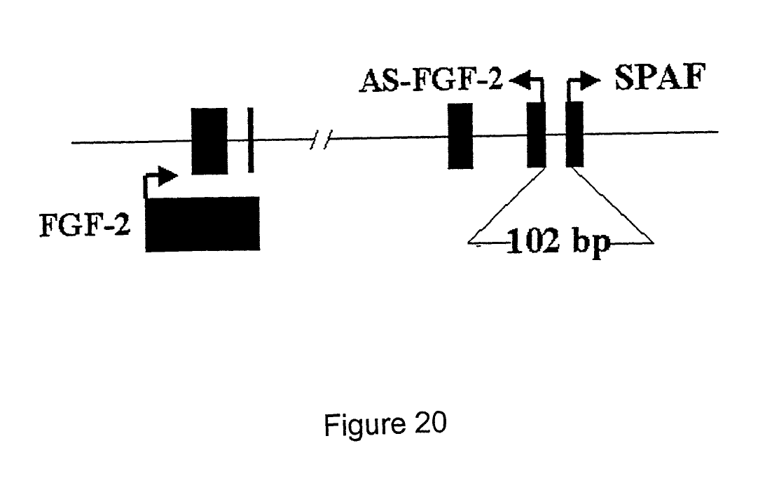

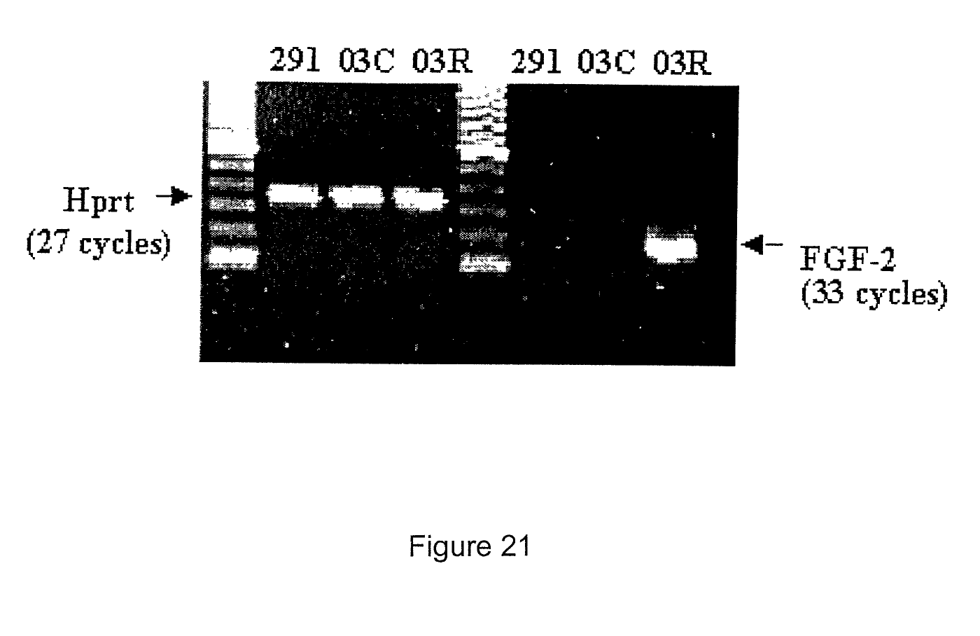

Our information from cloning the human SPAF gene also has enabled us to discover a unique promoter structure which exerts reciprocal control of SPAF and the FGF-2 gene expression. The novel SPAF gene sequence permitted identification of this structure within the human genome database, which is not well resolved in this region and does not properly align the SPAF gene. The human sequence draft match with our SPAF exon 1 indicate that this region is definitive. The major promoter region of the SPAF gene was found in the first exon of an adjacent gene, anti-sense FGF-2 (AS-FGF-2, accession #AF019632 and AF019633) whose transcript complements the 3′ untranslated region of basic fibroblast growth factor (FGF-2) (Gagnon et al., Journal of Cellular Biochemistry, 72:492-506, 1999). The first exon of SPAF lies in the immediate promoter region of AS-FGF-2. The first exons of SPAF and AS-FGF-2 are separated by an intergenic DNA of 102 bp. The major regulatory elements for each transcript reside in the first exon of the other (FIG. 20). Such closely related gene structure leads to competition for the direction of transcription. Orientation dependent transcription has been found in such bidirectional promoters (Guevara-Garcia et al., Molecular & General Genetics, 262:608-617, 1999). In this way, overexpression of SPAF in tumorigenic cells would result in downregulation of AS-FGF-2 in tumorigenic cells due to the bidirectional competition. Downregulation of AS-FGF-2 would result in upregulation of FGF-2 expression since the formation of double stranded RNA between FGF-2 and AS-FGF is one of the mechanisms for rapid RNA degradation (Kimelman et al., Cell, 59:687-696, 1989). To test this extrapolation, we examined FGF-2 expression in our epidermal model of carcinogenesis. Indeed, FGF-2 is overexpressed in the tumorigenic cells but not in non-cancerous precursor cells and FGF-2 expression mirrors SPAF expression (FIG. 21). FGF-2 is a potent growth stimulating factor for neuroectodermal and mesodermal cells. While FGF-2 expression is detected in a diverse variety of normal human tissues (Cordon-Cardo et al., Laboratory Investigation, 63:832-840, 1990), overexpression of FGF-2 is mainly detected in malignant melanoma and glioma (Basilico et al., Advances in Cancer Research, 59:115-165, 1992). As a matter of fact, FGF-2 expression is detected in virtually all the malignant melanoma tested (Shih et al., Journal of Investigative Dermatology, 100:196S-203S, 1993). The regulation of FGF-2 through the unique genomic structure suggests a role for SPAF overexpression in malignant melanoma development. There is potential for PSAF as a marker for melanoma prognosis. Of particular interest, the bi-directional promoter could be targeted as a molecular therapy to manipulate AS-FGF-2 expression and knock-out FGF-2 expression in melanoma therapy or other diseases exacerbated by FGF-2 overexpression.

TABLE 1
Amino Acid Sequence Similarity Between SPAF
and other AAA-Proteins

| | | % a.a. Identity | Length | Accession |
|---|---|---|---|---|
| Protein | Origin | (Similarity) | a.a. | No. |
| NSF | C. longicaudatus | 25 (50) | 752 | X15652 |
| hNSF | S. sapiens | 26 (51) | 742 | U03985 |
| PASI | S. cerevisiae | 27 (50) | 1043 | M58676 |
| SEC18 | S. cerevisiae | 29 (53) | 757 | M20662 |
| CDC48 | S. cerevisiae | 41 (64) | 834 | X56956 |
| p97ATPase | X. laevis | 42 (63) | 805 | X54240 |
| p97ATPase | R. norvegicus | 43 (65) | 806 | U11760 |
| mVCP | M. musculus | 43 (65) | 806 | Z14044 |
| AtCDC48 | A. thaliana | 43 (66) | 809 | U37587 |
| AFG2 | S. cerevisiae | 44 (64) | 780 | L14615 |
| SAV | S. acidocaldarius | 45 (66) | 780 | L17042 |
| SPAF | M. musculus | 100 (100) | 892 | |

TABLE 2
Eagle's MEM Medium

| Compound | mM | mg/liter |
|---|---|---|
| arginine | 0.6 | 105 |
| cystine | 0.1 | 24 |
| glutamine | 2.0 | 292 |
| histidine | 0.2 | 31 |
| isoleucine | 0.4 | 52 |
| leucine | 0.4 | 52 |
| lysine | 0.4 | 58 |
| phenylalanine | 0.2 | 32 |
| threonine | 0.4 | 48 |
| tryptophan | 0.05 | 10 |
| tyrosine | 0.2 | 36 |
| valine | 0.4 | 46 |
| glucose | 5.5 | 1000 |
| NaCl | 116 | 6800 |
| KCl | 5.4 | 400 |
| CaCl | 1.8 | 200 |
| MgCl₂ | 1.0 | 200 |
| NaH₂PO₄ | 1.1 | 150 |
| NaHCO₃ | 23.8 | 2000 |
| choline | | 1 |
| folic acid | | 1 |
| inositol | | 1 |
| nicotinamide | | 1 |
| pantothenate | | 1 |
| pyridoxal | | 1 |
| riboflavin | | 1 |
| thiamine | | 0.1 |

* From GIBCO/BRL

TABLE 3
Earle's Balanced Salt Solutions

| Component | gram/liter |
|---|---|
| sodium chloride | 6.8 |
| potassium chloride | 0.4 |
| calcium chloride | 0.2 |
| magnesium sulfate | 0.1 |
| sodium dihydrogen phosphate | 0.125 |
| sodium bicarbonate | 2.2 |
| glucose | 1.0 |

* From GIBCO/BRL

TABLE 4

| | | | Identity % |
|---|---|---|---|
| Gene Description | Accession # | Species | with hSPAF |
| valosin-containing protein | AAG29873 | Homo sapiens | 58.3 |
| Human N-ethylmaleimide- | U03985 | Homo sapiens | 39.7 |
| sensitive factor mRNA | | | |
| S. cerevisiae CDC48 gene | X56956 | S. cerevisiae | 53.7 |
| for cell cycle protein | | | |
| CDC48p | | | |
| M. musculus mRNA for | Z14044 | M. musculus | 55.5 |
| valosin-containing protein | | | |
| Mus musculus | Af049099 | M. musculus | 82.6 |
| spermatogenesis associated | | | |
| factor (SPAF) | | | |

What is claimed is:

1. A purified spermatogenesis associated factor DNA molecule having a DNA sequence, and being designated SPAF DNA which is a gene that is altered and overexpressed in poorly differentiated squamous carcinoma as compared with normal parental epidermal cells, said gene being naturally expressed in testes, said gene expressing a spermatogenesis associated protein by translation of said SPAF DNA molecule, said protein being designated SPAF protein, which is naturally localized in spermatogonia and primary spermatocytes up to early pachytene stage and which is ATPase associated with diverse cellular activities.

2. An SPAF cDNA molecule having a DNA sequence which is the complementary DNA sequence to the sequence of the SPAF DNA molecule of claim 1.

3. An anti-SPAF antibody.

4. A method for diagnosis of cancer by contacting a cell with a specific probe comprising SPAF cDNA and detecting a reaction.

5. A method for isolating SPAF gene comprising contacting the gene with SPAF cDNA.

6. A method for diagnosis of cancer comprising contacting a cancer cell with anti-SPAF antibody and detecting reaction to said antibody.

7. A method for diagnosing infertility comprising contacting testes generated cellular material with anti-SPAF antibody.

8. A method for isolation of SPAF gene by contacting the gene with anti-SPAF antibody.

9. A chemotherapeutic reagent comprising an antibody to SPAF protein.

10. A chemotherapeutic reagent comprising an antibody to SPAF protein conjugated with a tumor inhibiting material.

11. A vector containing a testes specific nucleic acid comprising at least a portion of the SPAF DNA molecular sequence of claim 1.

12. The vector of claim 11 comprising a bacteriophage.

13. The vector of claim 11, comprising a plasmid.

14. The SPAF DNA molecule of claim 1 in combination with an SPAF cDNA molecule having a DNA sequence that is complementary to the sequence of said SPAF DNA molecule.

15. The purified spermatogenesis associated factor DNA sequence, designated SPAF DNA as deposited on Oct. 17, 1997, in p Bluescript in E.coli HB101 with the American Type Culture Collection (ATCC) having ATCC deposit designation 98558.

16. An SPAF cDNA sequence which is the complementary DNA sequence to the SPAF DNA sequence of claim 15.

17. A vector containing a testes specific nucleic acid comprising at least a portion of the SPAF DNA sequence of claim 15.

18. The vector of claim 17 comprising a bacteriophage.

19. The vector of claim 17 comprising a plasmid.
